(19) United States
(12) Patent Application Publication
(10) Pub. No.: US 2002/0094525 A1
(43) Pub. Date: Jul. 18, 2002

(54) METHODS FOR THE DETECTION OF MULTIPLE SINGLE NUCLEOTIDE POLYMORPHISMS IN A SINGLE REACTION

(76) Inventors: TINA MCINTOSH, ELLICOTT CITY, MD (US); STEPHEN HEAD, HAMPSTEAD, MD (US); PHILIP GOELET, REISTERSTOWN, MD (US); MICHAEL T. BOYCE-JACINO, FINKSBURG, MD (US)

Correspondence Address: FRANKLIN S ABRAMS, KALOW SPRINGUT & BRESSLER LLP, 488 MADISON AVENUE, 19TH FLOOR, NEW YORK, NY, 10022, US

(21) Appl. No.: 09/454,394

(22) Filed: Dec. 3, 1999

Related U.S. Application Data
(63) Continuation of application No. 08/881,845, filed on Jun. 25, 1997, now abandoned.
(63) Continuation-in-part of application No. 08/216,538, filed on Mar. 23, 1994, now abandoned.
Application No. 08/216,538 is a continuation-in-part of application No. 08/145,145, filed on Nov. 3, 1993, now abandoned.

(51) Int. Cl. C12Q 1/68

(52) U.S. Cl. 435/6

(57) ABSTRACT

Molecules and methods suitable for identifying multiple polymorphic sites in the genome of a plant or animal. The identification of such sites is useful in determining identity, ancestry, predisposition to genetic disease, the presence or absence of a desired trait, etc.

### CROSS REFERENCE TO RELATED APPLICATIONS

This application is a continuation-in-part of U.S. application Ser. No. 08/216,538 (filed on Mar. 23, 1994) which is a continuation-in-part of U.S. application Ser. No. 08/145,145 (filed on Nov. 3, 1993).

### FIELD OF THE INVENTION

The present invention is in the field of recombinant DNA technology. More specifically, the invention is directed to molecules and methods suitable for identifying one or more single nucleotide polymorphisms in a single reaction in the genome of a plant, animal, or microorganism, and using such sites to analyze identity, ancestry or genetic traits.

1. Background of The Invention

The capacity to genotype an animal, plant or microbe is of fundamental importance to forensic science, medicine and epidemiology and public health, and to the breeding and exhibition of animals. Such a capacity is needed, for example, to determine the identity of the causative agent of an infectious disease, to determine whether two individuals are related, or to map genes within an organism's genome.

The analysis of identity and parentage, along with the capacity to diagnose disease is also of central concern to human, animal and plant genetic studies, particularly forensic or paternity evaluations, and in the evaluation of an individual's risk of genetic disease. Such goals have been pursued by analyzing variations in DNA sequences that distinguish the DNA of one individual from another.

If such a variation alters the lengths of the fragments that are generated by restriction endonuclease cleavage, the variations are referred to as restriction fragment length polymorphisms (“RFLPs”). RFLPs have been widely used in human and animal genetic analyses (Glassberg, J., UK patent Application 2135774; Skolnick, M. H. et al. Cytogen. Cell Genet. 32:58-67 (1982); Botstein, D. et al. Ann. T. Hum. Genet. 32:314331 (1980); Fischer, S. G. et al. (PCT Application W090/13668); Uhlen, M., PCT Application W090/11369)). Where a heritable trait can be linked to a particular RFLP, the presence of the RFLP in a target animal can be used to predict the likelihood that the animal will also exhibit the trait. Statistical methods have been developed to permit the multilocus analysis of RFLPs such that complex traits that are dependent upon multiple alleles can be mapped (Lander, S. etal. Proc. Natl. Acad. Sci. (U.S.A.) 83:7353-7357 (1986); Lander, S. et al., Proc. Natl. Acad. Sci. (U.S.A.) 84:2363-2367 (1987); Donis-Keller, H. et al Cell 51:319-337 (1987); Lander, S. et al., Genetics 121:185-199 (1989), all herein incorporated by reference). Such methods can be used to develop a genetic map, as well as to develop plants or animals having more desirable traits (Donis-Keller, H. etal. Cell 51:319-337 (1987); Lander, S. et al., Genetics 121:185-199 (1989)).

In some cases, the DNA sequence variations are in regions of the genome that are characterized by short tandem repeats (“STRs”) that include tandem di- or tri-nucleotide repeated motifs of nucleotides. These tandem repeats are also referred to as “variable number tandem repeat” (“VNTR”) polymorphisms. VNTRs have been used in identity and paternity analysis (Weber, J. L., U.S. Pat. No. 5,075,217; Armour, J. A. L. et al., FEBS Lett. 307:113-115 (1992); Jones, L. et al., Eur. I. Haematol. 39:144-147 (1987); Horn, G. T. et al. PCT Application W091/14003; Jeffreys, A. J., European Patent Application 370,719; Jeffreys, A. J., U.S. Pat. No. 5,175,082); Jeffreys, A. J. et al., Amer. T. Hum. Genet. 39:11-24 (1986); Jeffreys. A. J., et al., Nature 316:7679 (1985); Gray, I. C. et al., Proc. R. Acad. Soc. Lond. 243:241-253 (1991); Moore, S. S. et al., Genomics 10:654-660 (1991); Jeffreys, A. J. et al., Anim. Genet. 18:1-15 (1987); Hillel, J. et al., Anim. Genet. 20:145-155 (1989); Hillel, J. et al., Genet. 124:783-789 (1990)) and are now being used in a large number of genetic mapping studies.

A third class of DNA sequence variation results from single nucleotide polymorphisms (“SNPs”) that exist between individuals of the same species. Such polymorphisms are far more frequent than STRs and VNTRs. In some cases, such polymorphisms comprise mutations that are the determinative characteristic in a genetic disease. Indeed, such mutations may affect a single nucleotide in a protein-encoding gene in a manner sufficient to actually cause the disease (i.e. hemophilia, sickle-cell anemia, etc.). In many cases, these SNPs are in noncoding regions of a genome.

Despite the central importance of such polymorphisms in modern genetics, no practical method has been developed that permits the analysis of one or more loci from an individual in a single reaction format.

The present invention provides such an improved method. Indeed, the present invention provides methods and gene sequences that permit the genetic analysis of identity and parentage, and the diagnosis of disease by discerning the variation of multiple single nucleotide polymorphisms.

2. Summary of The Invention

The present invention is directed to molecules that comprise single nucleotide polymorphisms (SNPs) that are present in all life forms. The invention is directed to methods for (i) identifying one or more novel single nucleotide polymorphisms (ii) methods for the repeated analysis and testing of these SNPs in different samples and (iii) methods for exploiting the existence of such sites in the genetic analysis of animals, plants, and microbes.

The analysis (genotyping) of such sites is useful in determining identity, ancestry, predisposition to genetic disease, the presence or absence of a desired trait, etc. In detail, the invention provides one or more interrogation nucleic acid (or nucleic acid analog) primer molecules having a polynucleotide sequence complementary to one or more nucleotide sequences of a genomic DNA segment of any organism, the genomic segment being located immediately 3′-distal to a single nucleotide polymorphic site, X, of a single nucleotide polymorphic allele of the mammal; and wherein template-dependent extension of the nucleic acid (or nucleic acid analog) primer molecule by a single nucleotide (or nucleotide analog) extends the primer molecule by a single nucleotide, (or analog) the single nucleotide (or analog) being complementary to the nucleotide, X, of the single nucleotide polymorphic allele.

The invention concerns an embodiment wherein the template-dependent extension of the primer is conducted in the presence of one or more dideoxynucleotide triphosphate derivatives (or analogs) selected from the group consisting of ddATP, ddTTP, ddCTP and ddGTP (or other chain terminating base analogs), but in the absence of dATP, dT'TP, dCTP and dGTP.

The invention further provides a method for identifying one or more single nucleotide polymorphic sites in a single reaction which comprises the steps:

(A) hybridizing one or more of distinguishable interrogation oligonucleotide (or oligonucleotide analog) primers to one or more target nucleic acid molecules wherein each oligonucleotide primer is complementary to a specific and unique region of each target nucleic acid molecule such that the 3′ end of each primer is immediately proximal to a specific and unique target nucleotide of interest;

B) extending each interrogation oligonucleotide (or analog) with a template-dependent polymerase wherein said extension occurs in the presence of one or more non-extendible nucleotide (or nucleotide analog) species;

C) determining the identity of each nucleotide (or analog) of interest by determining, for each interrogation primer employed, the identity of the non-extendible nucleotide (or nucleotide analog) incorporated into such primer, said identified non-extendible nucleotide (or nucleotide analog) being complementary to said primer's target nucleotide; and

D) separating (or identifying) said extended primers on a suitable matrix, or by any other standard method of physical or chemical separation, or method of identification.

### BRIEF DESCRIPTION OF THE FIGURES

FIG. 1 illustrates the preferred method for cloning random genomic fragments. Genomic DNA is size fractionated, and then introduced into a plasmid vector, in order to obtain random clones. PCR primers are designed, and used to sequence the inserted genomic sequences.

FIG. 2 illustrates the data generated by the preferred method for identifying new polymorphic sequences which is cycle sequencing of a random genomic fragment.

FIG. 3 illustrates the RFLP method for screening random clones for polymorphic sequences.

FIG. 4 shows a graph of the probability that two individuals will have identical genotypes with given panels of genetic markers.

FIG. 5 shows a graph of the probability that given panels of 20 genetic markers will exclude a random alleged father in a paternity suit in which the mother is not in question.

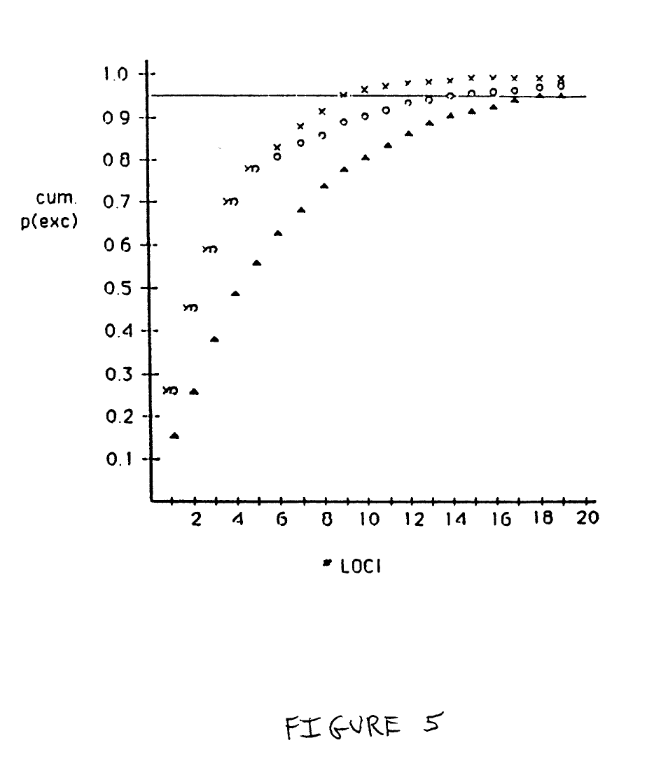

FIG. 6 illustrates the preferred method for genotyping SNPs. The seven steps illustrate how GBA can be performed starting with a biological sample.

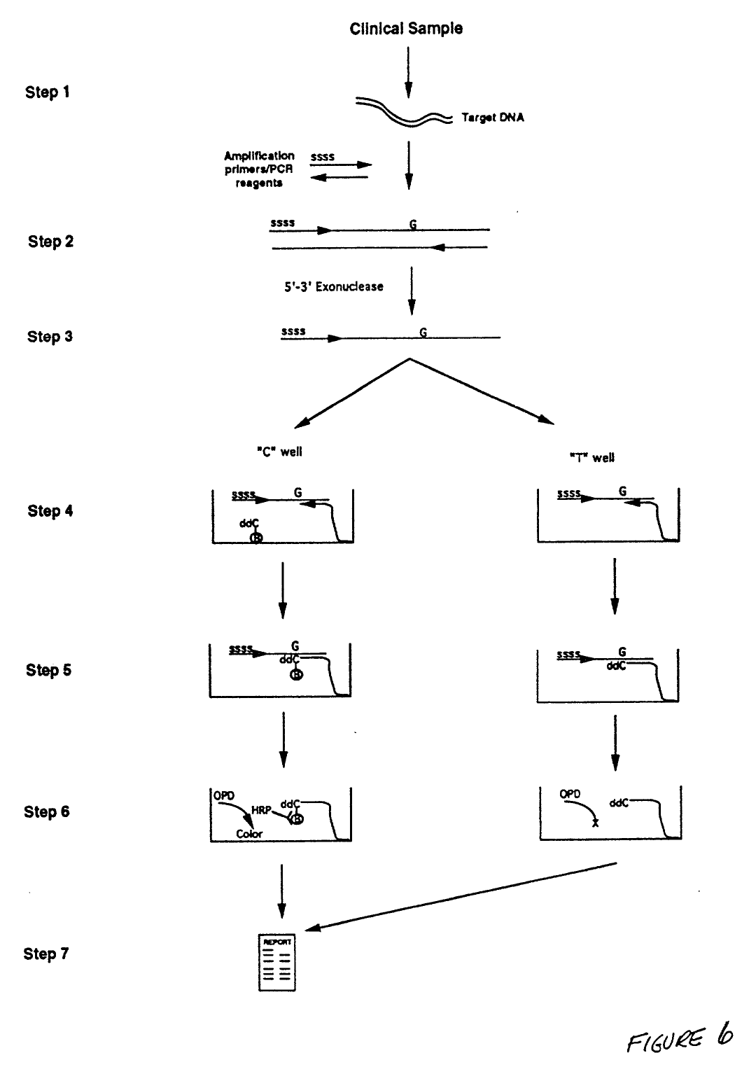

### DESCRIPTION OF THE PREFERRED EMBODIMENTS

I. The Single Nucleotide Polymorphisms of the Present Invention and the Advantages of their Use in Genetic Analysis

A. The Attributes of the Polymorphisms

The particular gene sequences of interest to the present invention comprise “single nucleotide polymorphisms.” A “polymorphism” is a variation in the DNA sequence of some members of a species. The genomes of animals and plants naturally undergo spontaneous mutation in the course of their continuing evolution (Gusella, J. F., Ann. Rev. Biochem. 55:831-854 (1986)). The majority of such mutations create polymorphisms. The mutated sequence and the initial sequence co-exist in the species' population. In some instances, such co-existence is in stable or quasi-stable equilibrium. In other instances, the mutation confers a survival or evolutionary advantage to the species, and accordingly, it may eventually (i.e. over evolutionary time) be incorporated into the genome of every member of that species.

A polymorphism is thus said to be “allelic,” in that, due to the existence of the polymorphism, some members of a species may have the unmutated sequence (i.e., the original “allele”) whereas other members may have a mutated sequence (i.e., the variant or mutant “allele”). In the simplest case, only one mutated sequence may exist, and the polymorphism is said to be diallelic. The occurrence of alternative mutations can give rise to triallelic polymorphisms, etc. An allele may be referred to by the nucleotide(s) that comprise the mutation.

The present invention is directed to a particular class of allelic polymorphisms, and to their use in genotyping plants, animals, or microbes. Such allelic polymorphisms are referred to herein as “single nucleotide polymorphisms,” or “SNPs.” “Single nucleotide polymorphisms” are defined by the following attributes. A central attribute of such a polymorphism is that it contains a polymorphic site, “X,” which is the site of variation between allelic sequences. A second characteristic of a SNP is that its polymorphic site “X” is frequently preceded by and followed by “invariant” sequences of the allele. The polymorphic site of the SNP is thus said to lie “immediately” 3′ to a “5′-proximal” invariant sequence, and “immediately” 5′ to a “3′-distal” invariant sequence. Such sequences flank the polymorphic site. The term “single” of single nucleotide polymorphisms refers to the number of nucleotides of the polymorphism (i.e. one nucleotide); it is unrelated to the number of polymorphisms present in the target DNA (which may range from one to many).

As used herein, a sequence is said to be an “invariant” sequence of an allele if the sequence does not vary in the population of the species, and if mapped, would map to a “corresponding” sequence of the same allele in the genome of every member of the species population. It should be noted that two or more SNP's may be very close in proximity to each other. Two sequences are said to be “corresponding” sequences if they are analogs of one another obtained from different sources. The gene sequences that encode hemoglobin in two humans illustrate “corresponding” allelic sequences. The definition of “corresponding alleles” provided herein is intended to clarify, but not to alter, the meaning of that term as understood by those of ordinary skill in the art. Each row of Table 1 shows the identity of the nucleotide of the polymorphic site of “corresponding” equine alleles, as well as the invariant 5′-proximal and 3′-distal sequences that are also attributes of that SNP. “Corresponding alleles” are illustrated in Table 2 with regard to human alleles. Each row of Table 2 shows the identity of the nucleotide of the polymorphic site of “corresponding” human alleles, as well as the invariant 5′-proximal and 3′-distal sequences that are also attributes of that SNP.

TABLE 1

| | POLYMORPHIC LOCI | | | | | |
|---|---|---|---|---|---|---|
| | SEQ | | IDENTIFIED SNP | | | |
| | ID | | ALLELE | | | SEQ ID |
| CLONE | NO. | 5′ PROXIMAL SEQUENCE | 1 | 2 | 3′ DISTAL SEQUENCE | NO. |
| 177-2 | 1 | GCAGCTCTAAGTGCTGTGGG | C | T | TGCAGAAATTCTAAGGTGTT | 2 |
| | 3 | AACACCTTAGAATTTCTGCA | G | A | CCCACAGCACTTAGAGCTGC | 4 |
| 595-3 | 5 | AGCTCTGGGATGATCCACTA | A | G | TGAGGGAAAAATGATGATGC | 6 |
| | 7 | GCATCATCATTTTTCCCTCA | T | C | TAGTGGATCATCCCAGAGCT | 8 |
| 090-2 | 9 | AAAACTAATTTGATGGCCAT | G | A | AAAGTCAGAACAATGATTGC | 10 |
| | 11 | GCAATCATTGTTCTGACTTT | C | T | ATGGCCATCAAATTAGTTTT | 12 |
| 324-1 | 13 | CACAAGGCCCAAGAACAGGA | T | C | TGAGTTCAGCGAGTGTCAGA | 14 |
| | 15 | TCTCACACTCGCTGAACTCA | A | G | TCCTGTTCTTGGGCCTTGTG | 16 |
| 129-1 | 17 | TGGGAAAGACCACATTATTT | T | A | GTTCCCTTTTGTTTCAGACC | 18 |
| | 19 | GGTCTGAAACAAAAGGGAAC | A | T | AAATAATGTGGTCTTTCCCA | 20 |
| 007-1 | 21 | CATGAGTAAGAAGCATCCGG | G | C | CCATGGAGTCATAGATAAGT | 22 |
| | 23 | ACTTATCTATGACTCCATGG | C | G | CCGGATGCTTCTTACTCATG | 24 |
| 324-2 | 25 | CCCAAGAACAGGATTGAGTT | C | T | AGCGAGTGTCAGAGTTGTGT | 26 |
| | 27 | ACACAACTCTGACACTCGCT | G | A | AACTCAATCCTGTTCTTCGG | 28 |
| 177-3 | 29 | AGCAAGAAATGGGGGGCCTT | A | G | GTCCTACAATTGCCAGGAAG | 30 |
| | 31 | CTTCCTGGCAATTGTAGGAC | T | C | AAGGCCCCCCATTTCTTGCT | 32 |
| 595-1 | 33 | GAATATCAATATATATATAT | G | A | TGTGTGTGTGTGTATTTGCT | 34 |
| | 35 | AGCAAATACACACACACACA | C | T | ATATATATATATTGATATTC | 36 |
| 007-3 | 37 | GCCATAATTAAGCCTGTATT | A | G | GTTTGTTTTAAATTTTGTGA | 38 |
| | 39 | TCACAAAATTTAAAACAAAC | T | C | AATACACGCTTAATTATGGC | 40 |
| 459-1 | 41 | GTGTAGAGTAGTTCAAGGAC | A | C | ATGTCTTATACCTCCCTTTT | 42 |
| | 43 | AAAAGGGAGGTATAAGACAT | T | G | GTCCTTGAACTACTCTACAC | 44 |
| 085-1 | 45 | GTGAACGGAGAGCAGGCCTT | C | G | CCTGCTGAAGCCTCAGACCG | 46 |
| | 47 | CGGTCTGAGGCTTCAGCAGG | G | C | AAGGCCTGCTCTCCGTTCAC | 48 |
| 007-2 | 49 | CTGCTCTTTAGACTATGACC | G | A | TCAACCTTGCATCATGAGCT | 50 |
| | 51 | AGCTCATGATGCAAGGTTGA | C | T | GGTCATAGTCTAAAGAGCAG | 52 |
| 474-1 | 53 | TTTGAGCTGGGACCTCAGTC | T | A | TCTCCTGCCTTTAGACTCGA | 54 |
| | 55 | TCGAGTCTAAAGGCAGGACA | A | T | GACTGACGTCCCAGCTCAAA | 56 |
| 178-1 | 57 | GAACCTCTGGGCCGTGGATA | A | G | TTGTTCAGAAGCACAGGTGA | 58 |
| | 59 | TCACCTGTGCTTCTGAACAA | T | C | TATCCACGGCCCAGAGGTTC | 60 |
| 595-2 | 61 | GTATTTGCTAGCTCTGGGAT | T | G | ATCCACTAATGAGGGAAAAA | 62 |
| | 63 | TTTTTCCCTCATTAGTGGAT | A | C | ATCCCAGAGCTAGCAAATAC | 64 |
| 177-1 | 65 | GAAGTTGTGGGACAGATGTG | C | A | AGAGATGCAGCTCTAAGTGC | 66 |
| | 67 | GCACTTAGAGCTGCATCTCT | G | T | CACATCTGTCCCACAACTTC | 68 |
| 459-2 | 69 | CCATGAGGAAGCCTCCACAA | C | G | GTCCCAATAGTCTGGGATTC | 70 |
| | 71 | GAATCCCAGACTATTGGGAC | G | C | TTCTGGAGGCTTCCTCATGG | 72 |

TABLE 2

| | | | | | | | | cum | |
|---|---|---|---|---|---|---|---|---|---|
| | Genotype 1 | Genotype 2 | Genotype 3 | | | | p(non- | p(non- | cum |
| LOCUS | PP (#) | PQ (#) | QQ (#) | p | q | p(exc) | exc) | exc) | p(exc) |
| 324-1 | CC (11) | CT (30) | TT (19) | 0.433 | 0.567 | 0.185 | 0.815 | 0.815 | 0.185 |
| 324-2 | CC (21) | CT (24) | TT (9) | 0.611 | 0.389 | 0.181 | 0.819 | 0.667 | 0.333 |
| 459-1 | AA (5) | AC (22) | CC (31) | 0.276 | 0.724 | 0.160 | 0.840 | 0.560 | 0.440 |
| 459-2 | CC (53) | CG (6) | GG (0) | 0.949 | 0.051 | 0.046 | 0.954 | 0.535 | 0.465 |
| 474-1 | AA (35) | AT (21) | TT (4) | 0.758 | 0.242 | 0.150 | 0.850 | 0.453 | 0.547 |
| 178-1 | AA (38) | AG (16) | GG (4) | 0.793 | 0.207 | 0.137 | 0.863 | 0.391 | 0.609 |
| 090-2 | AA (13) | AG (28) | GG (17) | 0.466 | 0.534 | 0.187 | 0.813 | 0.318 | 0.682 |
| 177-1 | AA (2) | AC (12) | CC (46) | 0.133 | 0.867 | 0.102 | 0.898 | 0.285 | 0.715 |
| 177-2 | CC (18) | CT (23) | TT (18) | 0.500 | 0.500 | 0.188 | 0.813 | 0.232 | 0.768 |
| 595-3 | AA (14) | AG (28) | GG (11) | 0.528 | 0.472 | 0.187 | 0.813 | 0.189 | 0.811 |
| 177-3 | AA (26) | AG (25) | GG (9) | 0.642 | 0.358 | 0.177 | 0.823 | 0.155 | 0.845 |
| 595-2 | GG (34) | GT (13) | TT (3) | 0.810 | 0.190 | 0.130 | 0.870 | 0.135 | 0.865 |
| 595-1 | AA (25) | AG (21) | GG (5) | 0.696 | 0.304 | 0.167 | 0.833 | 0.113 | 0.887 |
| 085-1 | CC (32) | CG (24) | GG (4) | 0.733 | 0.267 | 0.157 | 0.843 | 0.095 | 0.905 |
| 129-1 | AA (7) | AT (33) | TT (20) | 0.392 | 0.608 | 0.181 | 0.819 | 0.078 | 0.922 |
| 007-1 | AA (22) | CG (29) | GG (9) | 0.608 | 0.392 | 0.181 | 0.819 | 0.064 | 0.936 |
| 007-2 | AA (3) | AG (25) | GG (31) | 0.263 | 0.737 | 0.156 | 0.844 | 0.054 | 0.946 |
| 007-3 | AA (27) | AG (32) | GG (1) | 0.717 | 0.283 | 0.162 | 0.838 | 0.045 | 0.955 |

Since genomic DNA is double-stranded, each SNP can be defined in terms of either the plus strand or the minus strand. Thus, for every SNP, one strand will contain an immediately 5′-proximal invariant sequence and the other strand will contain an immediately 3′-distal invariant sequence. In the preferred embodiment, wherein each SNP's polymorphic site, “X,” is a single nucleotide, each strand of the double-stranded DNA of the SNP will contain both an immediately 5′-proximal invariant sequence and an immediately 3′-distal invariant sequence.

Although the preferred SNPs of the present invention involve a substitution of one nucleotide for another at the SNP's polymorphic site, SNPs can also be more complex, and may comprise a deletion of a nucleotide from, or an insertion of a nucleotide into, one of two corresponding sequences. For example, a particular gene sequence may contain an A in a particular polymorphic site in some animals, whereas in other animals a single or multiple base deletion might be present at that site. Although the preferred SNPs of the present invention have both an invariant proximal sequence and invariant distal sequence, SNPs may have only an invariant proximal or only an invariant distal sequence.

Nucleic acid molecules having a sequence complementary to that of an immediately 3′-distal invariant sequence of a SNP can, if extended in a “template-dependent” manner, form an extension product that would contain the SNP's polymorphic site. A preferred example of such a nucleic acid molecule is a nucleic acid molecule whose sequence is the same as that of a 5′-proximal invariant sequence of the SNP. “Template-dependent” extension refers to the capacity of a polymerase to mediate the extension of a primer such that the extended sequence is complementary to the sequence of a nucleic acid template. A “primer” is a single-stranded oligonucleotide (or oligonucleotide analog) or a single-stranded polynucleotide (or polynucleotide analog) that is capable of being extended by the covalent addition of a nucleotide (or nucleotide analog) in a “template-dependent” extension reaction. In order to possess such a capability, the primer must have a 3′-hydroxyl (or other chemical group suitable for polymerase mediated extension) terminus, and be hybridized to a second nucleic acid molecule (i.e. the “template”). A primer is composed of: (1) a unique sequence of 8 bases or longer complementary to a specific region of the target molecule such that the 3′ end of the primer is immediately proximal to a target nucleotide of interests, and (2) a 5′ tail composed of a neutral component of a specific and unique length, physical, or chemical characteristic. Most preferably, the complementary region of the primer is about 20 bases, however, primers of shorter or greater length may suffice. Typically, the complementary region of the primer is from about 12 bases to about 20 bases. The neutral component of the 5′ tail is any non-specific, nonhybridizing polymer or chemical group such as polyT, abasic residues, etc. A “polymerase” is an enzyme that is capable of incorporating nucleoside triphosphates (or appropriate analog) to extend a 3′-hydroxyl group of a nucleic acid molecule, if that molecule has hybridized to a suitable template nucleic acid molecule. Polymerase enzymes are discussed in Watson, J. D., In: Molecular Biology of the Gene, 3rd Ed., W. A. Benjamin, Inc., Menlo Park, Calif. (1977), which reference is incorporated herein by reference, and similar texts. Other polymerases such as the large proteolytic fragment of the DNA polymerase I of the bacterium E. coli. commonly known as “Klenow” polymerase, E. coli DNA polymerase I, and bacteriophage T7 DNA polymerase, may also be used to perform the method described herein. Nucleic acids having the same sequence as that of the immediately 3′ distal invariant sequence of a SNP can be ligated in a template dependent fashion to a primer that has the same sequence as that of the immediately 5′ proximal sequence that has been extended by one nucleotide in a template dependent fashion.

B. The Advantages of Using SNPs in Genetic Analysis

The single nucleotide polymorphic sites of the present invention can be used to analyze the DNA of any plant, animal, or microbe. Such sites are suitable for analyzing the genome of mammals, including humans, nonhuman primates, domestic animals (such as dogs, cats, etc.), farm animals (such as cattle, sheep, etc.) and other economically important animals. They may, however, be used with regard to other types of animals, plants, and microorganisms. SNPs have several salient advantages for use in genetic analysis over STRs and VNTRs.

First, SNPs occur at greater frequency (approximately 10-100 fold greater), and with greater uniformity than STRs and VNTRs. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., predisposition to cancer) reflects a mutation at a particular locus, then any polymorphism that is linked to the particular locus can be used to predict the probability that an individual will be exhibiting that trait.

The value of such a prediction is determined in part by the distance between the polymorphism and the locus. Thus, if the locus is located far from any repeated tandem nucleotide sequence motifs, VNTR analysis will be of very limited value. Similarly, if the locus is far from any detectable RFLP, an RFLP analysis would not be accurate. However, since the SNPs of the present invention are present approximately once every 300 bases in the mammalian genome, and exhibit uniformity of distribution, a SNP can, statistically, be found within 150 bases of any particular genetic lesion or mutation. Indeed, the particular mutation may itself be an SNP. Thus, where such a locus has been sequenced, the variation in that locus' nucleotide is determinative of the trait in question.

Second, SNPs are more stable than other classes of polymorphisms. Their spontaneous mutation rate is approximately 10⁻⁹, approximately 1,000 times less frequent than VNTRs. Significantly, VNTR-type polymorphisms are characterized by high mutation rates.

Third, SNPs have the further advantage that their allelic frequency can be inferred from the study of relatively few representative samples. These attributes of SNPs permit a much higher degree of genetic resolution of identity, paternity exclusion, and analysis of an animal's predisposition for a particular genetic trait than is possible with either RFLP or VNTR polymorphisms.

Fourth, SNPs reflect the highest possible definition of genetic information —nucleotide position and base identity. Despite providing such a high degree of definition, SNPs can be detected more readily than either RFLPs or VNTRs, and with greater flexibility. Indeed, the complimentary strand of the allele can be analyzed to confirm the presence and identity of any SNP because DNA is double-stranded.

The flexibility with which an identified SNP can be characterized is a salient feature of SNPs. VNTR-type polymorphisms, for example, are most easily detected through size fractionation methods that can discern a variation in the number of the repeats. RFLPs are most easily detected by size fractionation methods following restriction digestion.

In contrast, SNPs can be characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation.

The “Genetic Bit Analysis” (“GBA”) method disclosed by Goelet, P. et al. (WO92/15712, herein incorporated by reference), and discussed below, is a method for determining the identity of a nucleotide present at a single nucleotide polymorphic site. GBA is a method of polymorphic site interrogation in which the nucleotide sequence information surrounding the site of variation in a target DNA sequence is used to design an oligonucleotide primer that is complementary to the region immediately adjacent to, but not including, the variable nucleotide in the target DNA. The target DNA template is selected from the biological sample and hybridized to the interrogating primer. This primer is extended by a single labeled dideoxynucleotide (or analog) using a DNA polymerase in the presence of one or more chain terminating nucleoside triphosphate precursors (or suitable analogs).

Cohen, D. et al. (PCT Application W091/02087) describes another related method of genotyping wherein dideoxynucleotides are used to extend a single primer by a single nucleotide in order to determine the sequence at a desired locus. Dale et al. (PCT Application W090/09455) discloses a method for sequencing a “variable site” using a primer in conjunction with a single dideoxynucleotide species. The method of Dale et al. further discloses the use of multiple primers and the use of a separation element. Ritterband, M., etal. (PCT Application W095/17676) describes an apparatus for the separation, concentration and detection of such target molecules in a liquid sample. Cheeseman, P. C. (U.S. Pat. No. 5,302,509) describes a related method of determining the sequence of a single stranded DNA molecule. The method of Cheeseman employs fluorescently labeled 3′-blocked nucleotide triphosphates with each base having a different fluorescent label.

Wallace et al. (PCT Application W089/10414) describes multiple PCR procedures which can be used to simultaneously amplify multiple regions of a target by using allele specific primers. By using allele specific primers, amplification can only occur if a particular allele is present in a sample.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvänen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyrén, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvänen, A.-C., et al., Amer. J. Hum. Genet. 52:46-59 (1993)). Such a range of locus-specific signals could be more complex to interpret, especially for heterozygotes, compared to the simple, ternary (2:0, 1:1, or 0:2) class of signals produced by the GBA method. In addition, for some loci, incorporation of an incorrect deoxynucleotide can occur even in the presence of the correct dideoxynucleotide (Komher, J. S. et al., Nucl. Acids. Res. 17:7779-7784 (1989)). Such deoxynucleotide misincorporation events may be due to the Km of the DNA polymerase for the mispaired deoxy-substrate being comparable, in some sequence contexts, to the relatively poor Km of even a correctly base paired dideoxy- substrate (Kornberg, A., et al., In: DNA Replication, 2nd Edition, W. H. Freeman and Co., (1992); New York; Tabor, S. et al., Proc. Natl. Acad. Sci. (U.S.A.) 86:4076-4080 (1989)). This effect would contribute to the background noise in the polymorphic site interrogation.

In contrast to all such methods, the method of the present invention permits or greatly facilitates the determination of the nucleotides present at multiple SNPs.

II. Methods for Discovering Novel Polymorphic Sites

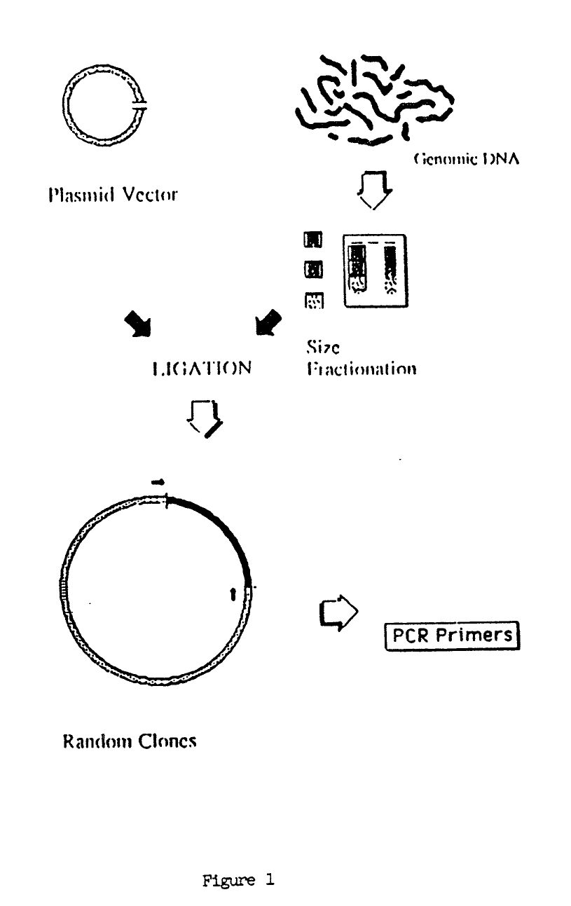

A preferred method for discovering polymorphic sites involves comparative sequencing of genomic DNA fragments from a number of haploid genomes. In a preferred embodiment, illustrated in FIG. 1, such sequencing is performed by preparing a random genomic library that contains 0.5-3 Kb fragments of DNA derived from one member of a species. Sequences of these recombinants are then used to facilitate PCR sequencing of a number of randomly selected individuals of that species at the same genomic loci.

From such genomic libraries (typically of approximately 50,000 clones), several hundred (200-500) individual clones are purified, and the sequences of the termini of their inserts are determined. Only a small amount of terminal sequence data (100-200 bases) need be obtained to permit PCR amplification of the cloned region. The purpose of the sequencing is to obtain enough sequence information to permit the synthesis of primers suitable for mediating the amplification of the equivalent fragments from genomic DNA samples of other members of the species. Preferably, such sequence determinations are performed using cycle sequencing methodology.

The primers are used to amplify DNA from a panel of randomly selected members of the target species. The number of members in the panel determines the lowest frequency of the polymorphisms that are to be isolated. Thus, if six members are evaluated, a polymorphism that exists at a frequency of, for example, 0.01 might not be identified. In an illustrative, but oversimplified, mathematical treatment, a sampling of six members would be expected to identify only those polymorphisms that occur at a frequency of greater than about 0.08 (i.e. 1.0 total frequency divided by 6 members divided by 2 alleles per genome). Thus, if one desires the identification of less frequent polymorphisms, a greater number of panel members must be evaluated.

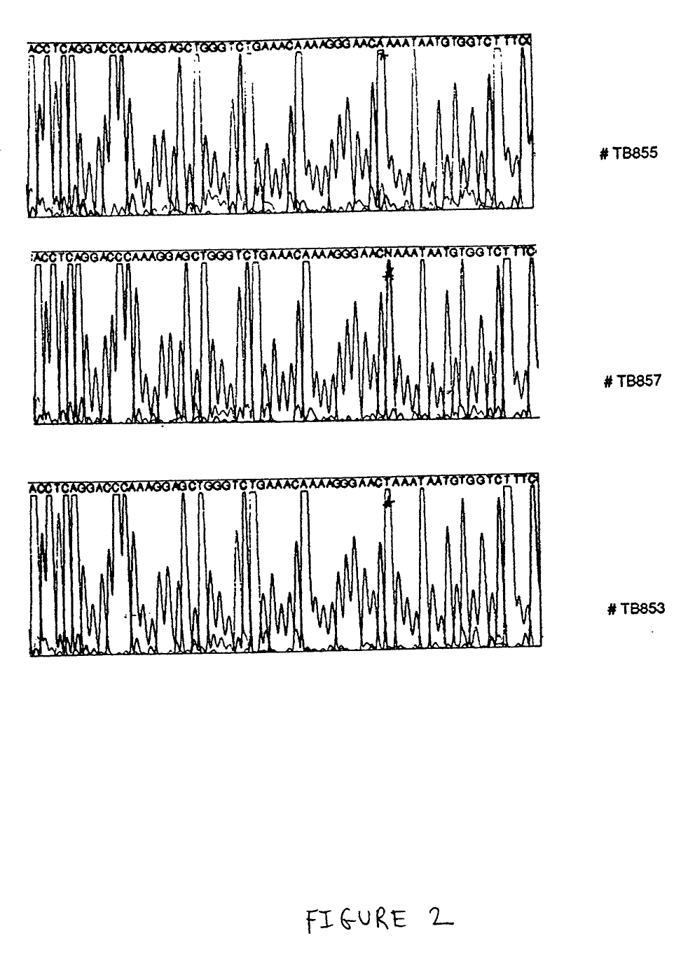

Cycle sequence analysis (Mullis, K. et al. Cold Spring Harbor Symp. Quant. Biol. 51:263-273 (1986); Erlich H. et al., European Patent Application 50,424; European Patent Application 84,796, European Patent Application 258,017, European Patent Application 237,362; Mullis, K., European Patent Application 201,184; Mullis K. et al., U.S. Pat. No. 4,683,202; Erlich, H., U.S. Pat. No. 4,582,788; and Saiki, R. et al. U.S. Pat. No. 4,683,194)) is facilitated through the use of automated DNA sequencing instruments and software (Applied Biosystems, Inc.). Differences between sequences of different animals can thereby be identified and confirmed by inspecting the relevant portion of the chromatograms on the computer screen. Differences are interpreted to reflect a DNA polymorphism only if the data was available for both strands, and present in more than one haploid example among the population of animals tested. FIG. 2 illustrates the preferred method for identifying new polymorphic sequences which is cycle sequencing of a random genomic fragment. The PCR fragments from the animal is electroeluted from acrylamide gels and sequenced using repetitive cycles of thermostable Taq DNA polymerase in the presence of a mixture of dNTPs and fluorescently or chemically labeled ddNTPs. The products are then separated and analyzed using an automated DNA sequencing instrument of Applied Biosystems, Inc. The data is analyzed using ABI software. Differences between sequences of different animals are identified by the software and confirmed by inspecting the relevant portion of the chromatograms on the computer screen. Differences are presented as “DNA Polymorphisms” only if the data is available for both strands and present in more than one haploid example among the five horses tested. The top panel shows an “A” homozygote, the middle panel an “AT” heterozygote and the bottom panel a “T” homozygote.

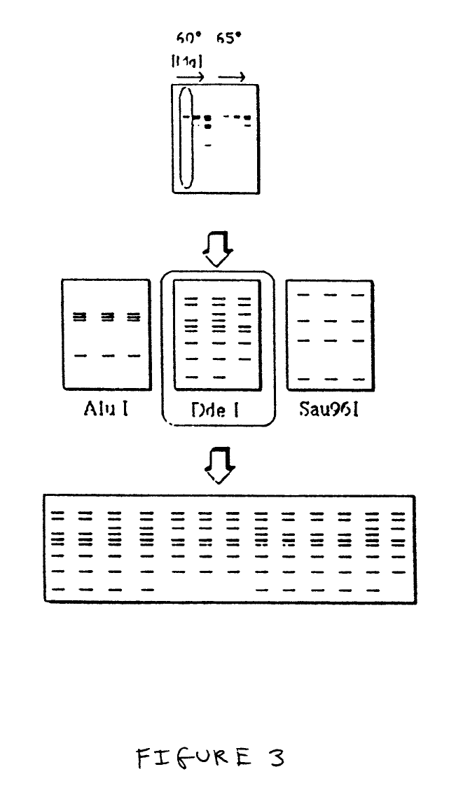

The discovery of polymorphic sites can alternatively be conducted using the strategy outlined in FIG. 3. In this embodiment, the DNA sequence polymorphisms are identified by comparing the restriction endonuclease cleavage profiles generated by a panel of several restriction enzymes on products of the PCR reaction from the genomic templates of unrelated members. Most preferably, each of the restriction endonucleases used will have four base recognition sequences, and will therefore allow a desirable number of cuts in the amplified products.

The restriction digestion patterns obtained from the genomic DNAs are preferably compared directly to the patterns obtained from PCR products generated using the corresponding plasmid templates. Such a comparison provides an internal control which indicates that the amplified sequences from the genomic and plasmid DNAs derive from equivalent loci. This control also allows identification of primers that fortuitously amplify repeated sequences, or multicopy loci, since these will generate many more fragments from the genomic DNA templates than from the plasmid templates.

III. Methods for Genotyping the Single Nucleotide Polymorphisms of the Present Invention

Any of a variety of methods can be used to identify the polymorphic site, “X,” of the single nucleotide polymorphisms of the present invention. The preferred method of such identification involves directly ascertaining the sequence of the polymorphic site for each polymorphism being analyzed. This approach is thus markedly different from the RFLP method which analyzes patterns of bands rather than the specific sequence of a polymorphism.

A. Amplification-Based Analysis

The detection of polymorphic sites in a sample of DNA may be facilitated through the use of DNA amplification methods. Such methods specifically increase the concentration of sequences that span the polymorphic site, or include that site and sequences located either distal or proximal to it. Such amplified molecules can be readily detected by gel electrophoresis or other means.

The most preferred method of achieving such amplification employs PCR, using primer pairs that are capable of hybridizing to the proximal sequences that define a polymorphism in its double-stranded form.

In lieu of PCR, alternative methods, such as the “Ligase Chain Reaction” (“LCR”) may be used (Barany, F., Proc. Natl. Acad. Sci. (U.S.A.) 25 88:189-193 (1991)). LCR uses two pairs of oligonucleotide probes to exponentially amplify a specific target. The sequences of each pair of oligonucleotides is selected to permit the pair to hybridize to abutting sequences of the same strand of the target. Such hybridization forms a substrate for a template-dependent ligase. As with PCR, the resulting products thus serve as a template in subsequent cycles and an exponential amplification of the desired sequence is obtained.

In accordance with the present invention, LCR can be performed with oligonucleotides having the proximal and distal sequences of the same strand of a polymorphic site. In one embodiment, either oligonucleotide will be designed to include the actual polymorphic site of the polymorphism. In such an embodiment, the reaction conditions are selected such that the oligonucleotides can be ligated together only if the target molecule either contains or lacks the specific nucleotide that is complementary to the polymorphic site present on the oligonucleotide.

In an alternative embodiment, the oligonucleotides will not include the polymorphic site, such that when they hybridize to the target molecule, a “gap” is created (see, Segev, D., PCT Application W090/01069). This gap is then “filled” with complementary dNTPs (as mediated by DNA polymerase), or by an additional pair of oligonucleotides. Thus, at the end of each cycle, each single strand has a complement capable of serving as a target during the next cycle and exponential amplification of the desired sequence is obtained.

The “Oligonucleotide Ligation Assay” (“OLA”) (Landegren, U. et al., Science 241:1077-1080 (1988)) shares certain similarities with LCR and may also be adapted for use in polymorphic analysis. The OLA protocol uses two oligonucleotides which are designed to be capable of hybridizing to abutting sequences of a single strand of a target. OLA, like LCR, is particularly suited for the detection of point mutations. Unlike LCR, however, OLA results in “linear” rather than exponential amplification of the target sequence.

Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Schemes based on ligation of two (or more) oligonucleotides in the presence of nucleic acid having the sequence of the resulting “di-oligonucleotide”, thereby amplifying the di-oligonucleotide, are (Wu, D. Y. et al., Genomics 4:560 (1989)), and may be readily adapted to the purposes of the present invention.

Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H.I. et al., PCT Application W089/06700; Kwoh, D. et al., Proc. Natl. Acad. Sci. (U.S.A.) 86:1173 (1989); Gingeras, T. R. et al., PCT Application WO088/10315)), or isothermal amplification methods (Walker, G. T. et al., Proc. Natl. Acad. Sci. (U.S.A.) 8:392-396 (1992)) may also be used.

B. Preparation of Single-Stranded DNA

The direct analysis of the sequence of SNPs in the present invention can be accomplished using either the “dideoxy-mediated chain termination method,” also known as the “Sanger Method” (Sanger, F., et al., J. Molec. Biol. 94:441 (1975)) or the “chemical degradation method,” also known as the “Maxam-Gilbert method” (Maxam, A. M., et al., Proc. Natl. Acad. Sci. (U.S.A.) 74:560 (1977), both references herein incorporated by reference). Methods for sequencing DNA using either the dideoxy-mediated method or the Maxam-Gilbert method are widely known to those of ordinary skill in the art. Such methods are disclosed, for example, in Sambrook, J., et al., Molecular Cloning, a Laboratory Manual, 2nd Edition, Cold Spring Harbor Press, Cold Spring Harbor, N.Y. (1989), and in Zyskind, J. W., et al., Recombinant DNA Laboratory Manual, Academic Press, Inc., New York (1988), both herein incorporated by reference.

Where a nucleic acid sample contains double-stranded DNA (or RNA), or where a double-stranded nucleic acid amplification protocol (such as PCR) has been employed, it is generally desirable to conduct such sequence analysis after treating the double-stranded molecules so as to obtain a preparation that is enriched for, and preferably predominantly, only one of the two strands. However, the generation of single stranded DNA template is not necessary for this invention if a thermo stable polymerase is used and the reaction is heated and cooled one or more times. This allows the double stranded template to separate from its complimentary strand and subsequently anneal to the interrogation primer(s) during the cooling step. Competition for hybridization by the other template strand can be compensated for by repeated cycling of the melting-cooling conditions.

The simplest method for generating single-stranded DNA molecules from double-stranded DNA is denaturation using either heat or alkali treatment. Single-stranded DNA molecules may also be produced using the single-stranded DNA bacteriophage M13 (Messing, J. et al., Meth. Enzymo1. 101:20 (1983); see also Sambrook, J., et al., (In: Molecular Cloning: A Laboratory Manual, Cold Spring Harbor Laboratory Press, Cold Spring Harbor, N.Y. (1989)).

Several alternative methods can be used to generate single-stranded DNA molecules. Gyllensten, U. et al., (Proc. Natl. Acad. Sci. (U.S.A.) 85:7652-7656 (1988) and Mihovilovic, M. et al., (BioTechniques 7(1):14 (1989)) describe a method, termed “asymmetric PCR,” in which the standard “PCR” method is conducted using primers that are present in different molar concentrations. Higuchi, R. G. et al., (Nucleic Acids Res. 17:5865 (1985)) exemplifies an additional method for generating single-stranded amplification products. The method entails phosphorolating the 5′-terminus of one strand of a double-stranded amplification product, and then permitting a 5′−>3′ exonuclease (such as T7 exonuclease) to preferentially degrade the phosphorylated strand.

Other methods have also exploited the nuclease resistant properties of phosphorothioate derivatives in order to generate single-stranded DNA molecules (Benkovic et al., U.S. Pat. No. 4,521,509; Sayers, J. R. et al., Nucl. Acids Res. 16:791-802 (1988); Eckstein, F. et al., Biochemistry 15:1685-1691 (1976); Ott, J. et al., Biochemistry 26:8237-8241 (1987)).

A discussion of the relative advantages and disadvantages of such methods of producing single-stranded molecules is provided by Nikiforov, T. (U.S. patent application Ser. No. 08/005,061 (application was abandoned Jun. 24, 1994), herein incorporated by reference).

In the most preferred embodiment, the phosphorothioate derivative is included in the primer. The nucleotide derivative may be incorporated into any position of the primer, but will preferably be incorporated at the 5′-terminus of the primer, most preferably adjacent to one another. Preferably, the primer molecules will have a complementary region approximately 25 nucleotides in length, and contain from about 4% to about 100%, and more preferably from about 4% to about 40%, and most preferably about 16%, phosphorothioate residues (as compared to total residues). The nucleotides may be incorporated into any position of the primer, and may be adjacent to one another, or interspersed across all or part of the primer.

In one embodiment, the present invention can be used in concert with an amplification protocol, for example, PCR. In this embodiment, it is preferred to limit the number of phosphorothioate bonds of the primers to about 10 (or approximately half of the length of the primers), so that the primers can be used in a PCR reaction without any changes to the PCR protocol that has been established for non-modified primers. When the primers contain more phosphorothioate bonds, the PCR conditions may require adjustment, especially of the annealing temperature, in order to optimize the reaction.

The incorporation of such nucleotide derivatives into DNA or RNA can be accomplished enzymatically, using a DNA polymerase (Vosberg, H. P. et al., Biochemistry 16: 3633-3640 (1977); Burgers, P. M. J. et al., J. Biol. Chem. 254:6889-6893 (1979); Kunkel, T. A., In: Nucleic Acids and Molecular Biology, Vol. 2, 124-135 (Eckstein, F. et al., eds.), Springer-Verlag, Berlin, (1988); Olsen, D. B. et al., Proc. Natl. Acad. Sci. (U.S.A.) 87:1451-1455 (1990); Griep, M. A. et al., Biochemistry 29:9006-9014 (1990); Sayers, J. R. et al., Nucl. Acids Res. 16:791-802 (1988)). Alternatively, phosphorothioate nucleotide derivatives can be incorporated synthetically into an oligonucleotide (Zon, G. et al., Anti-Canc. Drug Des. 6:539-568 (1991)).

The primer molecules are permitted to hybridize to a complementary target nucleic acid molecule, and are then extended, preferably via a polymerase, to form an extension product. The presence of the phosphorothioate nucleotides in the primers renders the extension product resistant to nuclease attack. As indicated, the amplification products containing phosphorothioate or other suitable nucleotide derivatives are substantially resistant to “elimination” (i.e., degradation) by “5′→3′” exonucleases such as T7 exonuclease or exonuclease, and thus a 5′→3′ exonuclease will be substantially incapable of further degrading a nucleic acid molecule once it has encountered a phosphorothioate residue.

Since the target molecule lacks nuclease resistant residues, the incubation of the extension product and its template —the target —in the presence of a 5′→3′ exonuclease results in the destruction of the template strand, and thereby achieves the preferential production of the desired single strand.

C. Hybridization of DNA in Solution

The preferred method of determining the identity of the polymorphic site of a polymorphism involves nucleic acid hybridization. Although such hybridization can be performed on a solid-phase (see, Saiki, R. K. et al., Proc. Natl. Acad. Sci. (U.S.A.) 86:6230-6234 (1989); Gilham et al., J. Amer. Chem. Soc. 86:4982 (1964) and Kremsky et al., Nucl. Acids Res. 15:3131-3139 (1987)), it is preferable to hybridize in solution (Berk, A. J., et al., Cell 12:721-732 (1977); Hood, L. E., et al., In: Molecular Biology of Eukaryotic Cells: A Problems Approach Menlo Park, Calif.: Benjamin-Cummings, (1975); Wetmer, J. G., Hybridization and Renaturation Kinetics of Nucleic Acids. Ann. Rev. Biophys. Bioeng. 5:337-361 (1976); Itakura, K., et al., Ann. Rev. Biochem. 53:323-356, (1984)).

For high volume testing applications, it is desirable to use nonradioactive detection methods. Thus, the use of fluorescently labeled or haptenated dideoxy-nucleotides is preferred. The use of biotinylated ddNTPs are preferably prepared by reacting the four respective (3-aminopropyn-1-yl) nucleoside triphosphates with sulfosuccinimidyl 6-(biotinamido)hexanoate. Thus, (3-aminopropyn-1-yl) nucleoside 5′-triphosphates are prepared as described by Hobbs, F. W. (J. Org. Chem. 54:3420-3422 (1989)) and by Hobbs, F. W. et al., (U.S. Pat. No. 5,047,519).

D. Analysis of Polymorphic Sites

1. Polymerase-Mediated Analysis

The identity of the nucleotide(s) of the polymorphic sites of the present invention can be determined, for example, using a variation of the oligonucleotide-based diagnostic assay of nucleic acid sequence variation disclosed by Goelet, P. et al., (PCT Application W092/15712, herein incorporated by reference). In particular, the present invention comprises an improvement over the method for analyzing SNPs described in U.S. patent application Ser. No. 08/216,538 (herein incorporated by reference), in that it permits or facilitates the simultaneous or nearly simultaneous analysis of multiple SNPs.

To accomplish such an advance, the present invention preferably employs one or more purified interrogation oligonucleotides having defined sequences that can hybridize to the target molecule in solution. The term “interrogation oligonucleotides” generally refers to oligonucleotide primers whose sequences are complementary to an immediate proximal or distal sequence of one or more single nucleotide polymorphisms.

In a preferred embodiment, one or more interrogation oligonucleotide primers having sequences that are complementary to specific regions of the target molecule are prepared using the above-described methods. Preferably, the primers have approximately 12 to 20 bases which are complementary to a specific region of the target molecule. The oligonucleotide primers hybridizes the target molecule such that the 3′ end of each primer is immediately proximal to a target nucleotide of interest (such as a SNP). Preferably, the oligonucleotide primer contains a 5′ tail composed of a neutral component (e.g., poly T, abasic residues, or other nonspecific, non-hybridizing polymer or chemical label). In the most preferred embodiment, the neutral component is assigned a specific and unique length. The primers may or may not contain a primer-specific label. However, in the most preferred embodiment, the oligonucleotide primers do contain a primer-specific label.

The interrogation primers are then incubated in the presence of the target DNA molecule (preferably a genomic DNA molecule) having one or more single nucleotide polymorphisms where the immediately 3′ distal sequence for each SNP is complementary to that of the interrogation primer, a DNA polymerase and a chain terminating nucleotide (or nucleotide analog) triphosphate derivative. Preferably, such incubation occurs in the complete absence of any dNTP (i.e. dATP, dGTP, dCTP, dTTI?), but only in the presence of one or more chain terminating nucleotide triphosphate derivatives (or analogs) (e.g., ddATP, ddGTT, ddCTT, ddTIP, etc.), and under conditions sufficient to permit a single base incorporation of the derivative onto the 3′ terminus of the primer. While the presence of unincorporated nucleotide triphosphate(s) in the reaction is immaterial to the reaction, such unincorporated nucleotides may be separated by a number of means. The identity of the incorporated nucleotide is determined by and is complementary to, the nucleotide of the polymorphic site of the polymorphism.

In the present invention, the non-extendible nucleotide may be labeled, preferably with ³²P or a florescent molecule. Other labels suitable for the present invention include, but are not limited to, biotin, iminobiotin, hapten, an antigen, a cofactor, dintrophenol, lipoic acid, an olefinic compound, a detectable polypeptide, a molecule that is electron dense, an enzyme capable of depositing an insoluble reaction product. Florescent molecules suitable for the present invention include, but are not limited to, fluorescein, rhodamine, texas red, FAM, JOE, TAMRA, ROX, HEX, TET, Cy3, Cy3.5, Cy5, Cy 5.5, IRD40, IRD41 and BODIPY. Electron dense indicator molecules suitable for the present invention include, but are not limited to, ferritin, hemocyanin and colloidal gold. The detectable polypeptide may be indirectly detectable by specifically complexing the detectable polypeptide with a second polypeptide covalently linked to an indicator molecule. In such an embodiment, the detectable polypeptide is preferably selected from the group consisting of avidin and strepavidin, and the second polypeptide is preferably selected from the group consisting of biotin and iminobiotin.

In a preferred embodiment, the resultant extended primers are separated for analysis on a suitable matrix. Any of a number of methods can be used to separate the extended primers for analysis. Such methods include, but are not limited to: mass spectrometry, (oligonucleotide array hybridization) flow cytometry, HPLC, FPLC, size exclusion chromatography, affinity chromatography, gel electrophoresis, etc. Preferably, the extended primers are separated under denaturing conditions; however, denaturing conditions are not required for effective separation.

The term non-extendible nucleotide refers to a synthetic or naturally occurring nucleotide analog that is capable of being incorporated by a template dependent polymerase. Synthetic or naturally occurring nucleotide analogs suitable for use in the present invention include, but are not limited to, acyclic ribose nucleotide analogs, substituted ribose nucleotide analogs, and modified ribose nucleotide analogs. Synthetic nucleotide analogs are preferably selected from the group consisting of fructose based nucleotide analogs, chemically modified purines that retain the ability to specifically base pair with naturally occurring nucleotides, chemically modified pyrimidines that retain the ability to specifically base pair with naturally occurring nucleotides, and any compound that retains the ability to specifically base pair with naturally occurring nucleotides.

In a most preferred embodiment, the extended primers are separated on a denaturing, size separating matrix such as a standard sequencing gel having an appropriate acrylamide concentration. This embodiment employs interrogation primers containing a 5′ tail having a specific and unique length. The extended primers are differentially separated based upon the specific and unique length of the 5′ tail. While the preferred embodiment employs labeled chain terminating nucleotide(s) (or nucleotide analog(s)), the present embodiment is also directed towards differentially labeled interrogation primers. One sub-embodiment employs differentially labeled chain terminating nucleotides (i.e., dideoxynucleotides). An alternate sub-embodiment employs one or more chain terminating nucleotides wherein only a single chain terminating nucleotide is labeled.

Another preferred embodiment employs interrogation primers containing unique and specific sequences capable of hybridizing to complimentary sequences arrayed on a solid phase. In this embodiment, the interrogation primers are separate by exposing them to arrayed capture primers and identifying each single nucleotide polymorphism through detection of the labeled base in the context of its location on the solid phase capture primer array.

In an alternate embodiment, the resultant extended primers are separated using suitable affinity separation methods. Such affinity separation methods are generally drawn to receptor-ligand methods (e.g., avidin-strepavidin, etc.), monoclonal antibody methods, etc. In this embodiment, the 5′ terminus of each primer is coupled to a unique ligand for which there is a corresponding unique receptor. In this embodiment, the receptors are preferably immobilized to a solid surface (i.e., a bead, a column, a dipstick, a microtiter plate, etc.). The ligand-labeled primers are separated by exposing the reaction mixture to the corresponding receptors. It is then possible to determine the identity of each single nucleotide polymorphism using the methods described above.

Another embodiment employs primers coupled (covalently or otherwise) to uniquely sized moieties (e.g., BSA, lysozyme, ovalbumin, etc.). In this embodiment, the uniquely sized primers are separated by passing the primers through a suitable size exclusion chromatography column and the identity of each single nucleotide polymorphism is identified using the methods described above.

It is also possible with the present invention to use the above-mentioned methods in combination to thereby increase the number SNPs that can be identified in a single reaction.

Labels suitable for use in the present invention include, but are not limited to: enzymes (β-galactosidase, luciferase, etc.), radioactive isotopes (i.e., ³²P, ¹³C, ³H, etc.), fluorescent moieties (i.e., fluorescein, rhodamine, etc.), chromophores. The primers can be either directly labeled or coupled with a distinct ligand which may be either labeled or unlabeled. The ligand molecule may be coupled to the oligonucleotide primer by covalent coupling, ionic interactions, non-specific adsorption, or specific but non-covalent ligand-receptor interactions.

The term ligand refers generally to a given protein or chemical compound to which there is a corresponding distinct receptor. Ligands suitable for use in the present invention include, but are not limited to, a hapten, an antigen, a cofactor, biotin, iminobiotin, dinitrophenol, lipoic acid, an olefinic compound, an oligonucleotide, protein nucleic acid (“PNA”) sequences designed to hybridize specifically to a complementary oligonucleotide, and PNA sequences that functions as a receptor. Additional ligands suitable for use in the present invention include, but are not limited to, an antibody, an enzyme, a polypeptide, strepavidin and avidin. In one embodiment, the ligand is capable of forming a complex by binding with a detectably labeled polypeptide. The detectable label suitable for use in the present invention includes, but is not limited to, an antibody, an enzyme capable of depositing insoluble reaction products, strepavidin and avidin. Preferably, the detectably labeled polypeptide is selected from randomly generated polypeptide libraries.

The term receptor refers generally to a given protein or chemical compound to which there is a corresponding ligand. Receptors suitable for use in the present invention include, but are not limited to, an antigen, a cofactor, biotin, iminobiotin, dinitrophenol, lipoic acid, an olefinic compound, an oligonucleotide, PNA sequences designed to hybridize specifically to a complementary oligonucleotide, and PNA sequences that functions as a ligand. In one embodiment, the receptor is capable of forming a complex by binding with a detectably labeled polypeptide. The detectable label suitable for use in the present invention includes, but is not limited to, an antibody, an enzyme capable of depositing insoluble reaction products, strepavidin and avidin. Preferably, the detectably labeled polypeptide is selected from randomly generated polypeptide libraries. The receptor may be coupled to the matrix. Suitable methods for coupling the receptor to the matrix include, but are not limited to, covalent coupling, ionic interactions, non-specific adsorption, specific but non-covalent ligand-receptor interactions. The ligand-receptor suitable for use in the present invention includes, but is not limited to, complementary hybridizing nucleic acids, complementary hybridizing PNAs, and other complementary synthetic nucleic acid analogs.

Although in the preferred embodiment the extended primers are separated prior to detection, the present invention can also be used to identify SNPs without separation of the primers. In this embodiment, at least one added chain terminating nucleotide triphosphate derivative is uniquely labeled, such that the addition of a nucleotide to an interrogation primer can be detected (either by the labeling of the oligonucleotide, or the failure of the oligonucleotide to become labeled). Thus, for example, if three primers are employed to interrogate three different SNPs in the presence of labeled ddATP, the incorporation of such label is indicative that one of the SNPs is a T.

The identification of the primers through the primer specific label and the incorporated nucleotides enables the genotyping of the target molecule. The nucleotide of the polymorphic site is thus determined by assaying which of the set of labeled nucleotides has been incorporated into the 3′ terminus of the oligonucleotide by the primer-dependent polymerase. The non-extendible nucleotide or nucleotide analog may be identified by any of a number of physical or chemical method. However, the preferred physical or chemical means are selected from the group consisting of polarization spectroscopy, mass spectroscopy, infra-red spectroscopy, ultra-violet spectroscopy, visible spectroscopy or NMR spectroscopy.

While the present method is directed at methods to identify multiple SNPs in a single reaction, the present invention can also confirm the identity of multiple SNPs in a single reaction. In this embodiment, the identity of each SNP for both the plus and minus strand of the target nucleic acid are determined as previously described. The sequence is confirmed where the plus and minus strand for each SNP analyzed are complementary.

2. Polymerase/Ligase-Mediated Analysis

In an alternative embodiment, the identity of the nucleotide of the polymorphic site is determined using a polymerase/ligase mediated process. As in the previous embodiments, multiple oligonucleotide primers are simultaneously employed for the detection of multiple SNPs in the same reaction.

As in the above described embodiments, an oligonucleotide primer is employed that is complementary to an immediately 3′ distal invariant sequence of a SNP. A second oligonucleotide, complementary to the 5′-proximal sequence of the polymorphism being analyzed, but incapable of hybridizing to the oligonucleotide primer is used.

These oligonucleotides are incubated in the presence of DNA containing the single nucleotide polymorphism that is to be analyzed, and at least one 2′, 5′-deoxynucleotide triphosphate. The incubation reaction further includes a DNA polymerase and a DNA ligase.

Both oligonucleotides are thus capable of hybridizing to the same strand of the single nucleotide polymorphism being analyzed. Sequence considerations cause the two oligonucleotides to hybridize to the proximal and distal sequences of the SNP that flank the polymorphic site (X) of the polymorphism; the hybridized oligonucleotides are thus separated by a “gap” of a single nucleotide at the precise position of the polymorphic site.

The presence of a polymerase and a 2′, 5′-deoxynucleotide triphosphate complementary to (X) permits ligation of the primer extended with the complementary 2′, 5′-deoxynucleotide triphosphate to the hybridized oligo complementary to the distal sequence, a 2′, 5′-deoxy-nucleotide triphosphate that is complementary to the nucleotide of the polymorphic site permits the creation of a ligatable substrate.

The identity of the polymorphic site that was opposite the “gap” can then be determined by any of several means. In a preferred embodiment, the 2′, 5′-deoxynucleotide triphosphate of the reaction is labeled, and its detection thus reveals the identity of the complementary nucleotide of the polymorphic site. Several different 2′, 5′-deoxynucleotide triphosphates may be present, each differentially labeled. Alternatively, separate reactions can be conducted, each with a different 2′, 5′-deoxynucleotide triphosphate. In an alternative sub-embodiment, the 2′, 5′-deoxynucleotide triphosphates are unlabeled, and the second, soluble oligonucleotide is labeled. Separate reactions are conducted, each using a different unlabeled 2′, 5′-deoxynucleotide triphosphate.

While the above-described embodiment details a polymerase/ligase mediated method for the detection of a single polymorphic site, it is generally understood that the method can employ the simultaneous use of multiple unique oligonucleotide primers for the detection of multiple polymorphic sites.

E. Signal-Amplification

The sensitivity of nucleic acid hybridization detection assays may be increased by altering the manner in which detection is reported or signaled to the observer. Thus, for example, assay sensitivity can be increased through the use of detectably labeled reagents. A wide variety of such signal amplification methods have been designed for this purpose. Kourilsky et al., (U.S. Pat. No. 4,581,333) describe the use of enzyme labels to increase sensitivity in a detection assay. Fluorescent labels (Albarella et al., EP 144914), chemical labels (Sheldon III et al., U.S. Pat. No. 4,582,789; Albarella et al., U.S. Pat. No. 4 ,563,417 ), modified bases (Miyoshi et al., EP 119448), etc. have also been used in an effort to improve the efficiency with which hybridization can be observed.

It is preferable to employ fluorescent, or chromogenic (especially enzyme) labels, such that the identity of the incorporated nucleotide can be determined in an automated, or semi-automated manner using appropriate detection instrumentation.

IV. The Use of SNP Genotyping in Methods of Genetic Analysis

A. General Considerations for Using Single Nucleotide Polymorphisms in Genetic Analysis

The utility of the polymorphic sites of the present invention stems from the ability to use such sites to predict the statistical probability that two individuals will have the same alleles for any given polymorphisms.

Statistical analysis of SNPs can be used for any of a variety of purposes. Where a particular individual has been previously tested, such testing can be used as a “fingerprint” which can be used to determine the identity of a particular individual.

Where a putative parent or both parents of an individual have been tested, the methods of the present invention may be used to determine the likelihood that a particular animal is or is not the progeny of such parent or parents. Thus, the detection and analysis of SNPs can be used to exclude paternity of a male for a particular individual (such as a father's paternity of a particular child), or to assess the probability that a particular individual is the progeny of a selected female (such as a particular child and a selected mother).

As indicated below, the present invention permits the construction of a genetic map of a target species. Thus, the particular array of polymorphisms identified by the methods of the present invention can be correlated with a particular trait, in order to predict the predisposition of a particular animal (or plant) to such genetic disease, condition, or trait. As used herein, the term “trait” is intended to encompass “genetic disease,” “condition,” or “characteristics.” The term, “genetic disease” denotes a pathological state caused by a mutation, regardless of whether that state can be detected or is asymptomatic. A “condition” denotes a predisposition to a characteristic (such as asthma, weak bones, blindness, ulcers, cancers, heart or cardiovascular illnesses, skeleto-muscular defects, etc.). A “characteristic” is an attribute that imparts economic value to a plant or animal. Examples of characteristics include longevity, speed, endurance, rate of aging, fertility, etc.

B. Identification and Parentage Verification

The most useful measurements for determining the power of an identification and paternity testing system are: (i) the “probability of identity” (p(ID)) and (ii) the “probability of exclusion” (p(exc)). The p(ID) calculates the likelihood that two random individuals will have the same genotype with respect to a given polymorphic marker. The p(exc) calculates the likelihood, with respect to a given polymorphic marker, that a random male will have a genotype incompatible with him being the father in an average paternity case in which the identity of the mother is not in question. Since single genetic loci, including loci with numerous alleles such as the major histocompatibility region, rarely provide tests with adequate statistical confidence for paternity testing, a desirable test will preferably measure multiple unlinked loci in parallel. Cumulative probabilities of identity or non-identity, and cumulative probabilities of paternity exclusion are determined for these multi-locus tests by multiplying the probabilities provided by each locus.

The statistical measurements of greatest interest are: (i) the cumulative probability of non-identity (cum p(nonID)), and (ii) the cumulative probability of paternity exclusion (cum p(exc)).

The formulas used for calculating these probability values are given below. For simplicity these are given first for 2-allele loci, where one allele is termed type A and the other type B. In such a model, four genotypes are possible: AA, AB, BA, and BB (types AB and BA being indistinguishable biochemically). The allelic frequency is given by the number of times A (f(A), the frequency of A is denoted by “p”) or B (f(B), the frequency of B is denoted by “q,” where q=1-p) is found in the haploid genome. The probability of a given genotype at a given locus:

Homozygote: p(AA)=p²

Single Heterozygote: p(AB)=p(BA)=pq =p(1−p)

Both Heterozygotes: p(AB+BA)=2pq=2p(1−p)

Homozygote: p(BB)=q²(1−p)²

The probability of identity at one locus (i.e. the probability that two individuals, picked at random from a population will have identical genotypes at a given locus) is given by the equation:

p(ID)=(p²)²+(2pq)²+(q²)²

The cumulative probability of identity for n loci is therefore given by the equation:

cum p(ID)=\tilde{ο}p(ID₁)p(ID₂)p(ID₃)p(IDₙ)

The cumulative probability of non-identity for n loci (i.e. the probability that two individuals will be different at 1 or more loci) is given by the equation:

cum p(nonID)=1−cum p(ID)

The probability of parentage exclusion (representing the probability that a random male will have a genotype, with respect to a given locus, that makes him incompatible as the sire in an average paternity case where the identity of the mother is not in question) is given by the equation:

p(exc)=pq(1−pq)

The probability of non-exclusion (representing the probability at a given locus that a random male will not be biochemically excluded as the sire in an average paternity case) is given by the equation:

p(non-exc)=1 −p(exc)

The cumulative probability of non-exclusion (representing the value obtained when n loci are used) is thus:

cum p(non−exc)=\tilde{ο}p(non-exc₂)p(non−exc₂)p(non−exc₃) p(non−excₙ)

The cumulative probability of exclusion (representing the probability, using a panel of n loci, that a random male will be biochemically excluded as the sire in an average paternity case where the mother is not in question) is given by the equation:

cum p(exc)=1−cum p(non−exc) These calculations may be extended for any number of alleles at a given locus. For example, the probability of identity p(ID) for a 3-allele system where the alleles have the frequencies in the population of p, q and r, respectively, is equal to the sum of the squares of the genotype frequencies:

p(ID)=p⁴+(2pq)²+(2qr)²+(2pr)²+r⁴+q⁴

Similarly, the probability of exclusion for a three allele system is given by:

p(exc)=pq(1−pq)+qr(1−qr)+pr(1−pr) +3pqr(1−pqr)

In a locus of n alleles, the appropriate binomial expansion is used to calculate p(ID) and p(exc).

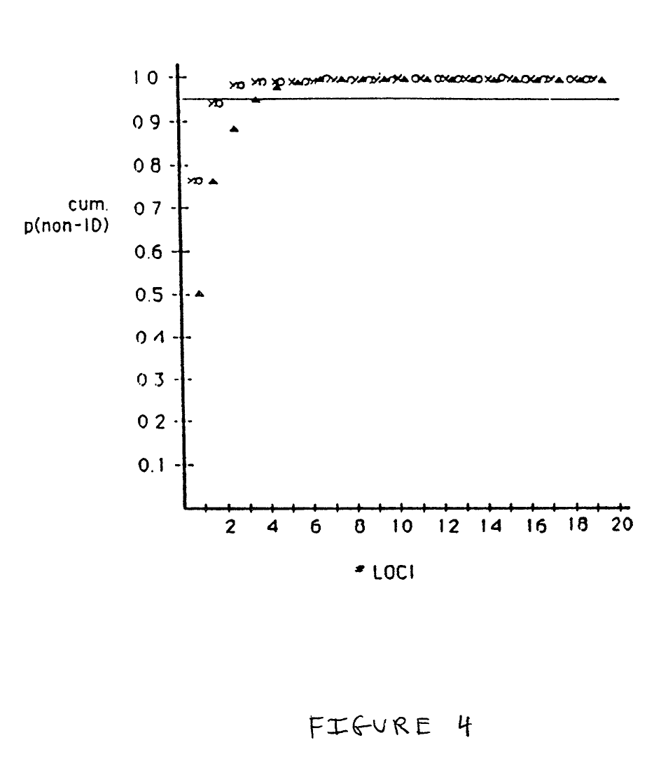

FIGS. 3 and 4 show how the cum p(nonID) and the cum p(exc) increase with both the number and type of genetic loci used. It can be seen that greater discriminatory power is achieved with fewer markers when using three allele systems. In FIGS. 3 and 4, the triangles trace the increase in probability values with increasing numbers of loci with two alleles where the common allele is present at a frequency of p=0.79. The crosses in FIGS. 3 and 4 show the same analysis for increasing numbers of three-allele loci where p=0.51, q=0.34 and r=0.15.

The choice between whether to use loci with 2, 3 or more alleles is, however, largely influenced by the above-described biochemical considerations. A polymorphic analysis test may be designed to score for any number of alleles at a given locus. If allelic scoring is to be performed using gel electrophoresis, each allele should be easily resolvable by gel electrophoresis. Since the length variations in multiple allelic families are often small, human DNA tests using multiple allelic families include statistical corrections for mistaken identification of alleles. Furthermore, although the appearance of a rare allele from a multiple allelic system may be highly informative, the rarity of these alleles makes accurate measurements of their frequency in the population extremely difficult. To correct for errors in these frequency estimates when using rare alleles, the statistical analysis of this data must include a measure of the cumulative effects of uncertainty in these frequency estimates. The use of these multiple allelic systems also increases the likelihood that new or rare alleles in the population will be discovered during the course of large population screening. The integrity of previously collected genetic data would be empirically revised to reflect the discovery of a new allele.

In view of these considerations, although the use of loci with many alleles could potentially offer some short-term advantages (because fewer loci would need to be screened), it is preferable to perform polymorphic analyses using loci with fewer alleles that are: (i) more frequently represented, and (ii) easier to measure unambiguously. Tests of this type can achieve the same power of discrimination as tests based on more highly polymorphic loci, provided the same total number of alleles is collected from a series of unlinked loci.

C. Gene Mapping and Genetic Trait Analysis Using SNPs

The polymorphisms detected in a set of individuals of the same species (such as humans, horses, etc.), or of closely related species, can be analyzed to determine whether the presence or absence of a particular polymorphism correlates with a particular trait.

To perform such polymorphic analysis, the presence or absence of a set of polymorphisms (i.e. a “polymorphic array”) is determined for a set of individuals, some of which exhibit a particular trait, and some of which exhibit a mutually exclusive characteristic (for example, with respect to horses, brittle bones vs. non-brittle bones; maturity onset blindness vs. no blindness; predisposition to asthma, cardiovascular disease, etc. vs. no such predisposition). The alleles of each polymorphism of the set are then reviewed to determine whether the presence or absence of a particular allele is associated with the particular trait of interest. Any such correlation defines a genetic map of the individual's species. Alleles that do not segregate randomly with respect to a trait can be used to predict the probability that a particular animal will express that characteristic. For example, if a particular polymorphic allele is present in only 20% of the members of a species that exhibit a cardiovascular condition, then a particular member of that species containing that allele would have a 20% probability of exhibiting such a cardiovascular condition. As indicated, the predictive power of the analysis is increased by the extent of linkage between a particular polymorphic allele and a particular characteristic. Similarly, the predictive power of the analysis can be increased by simultaneously analyzing the alleles of multiple polymorphic loci of a particular trait. In the above example, if a second polymorphic allele was found to also be present in 20% of members exhibiting the cardiovascular condition, however, all of the evaluated members that exhibited such a cardiovascular condition had a particular combination of alleles for these first and second polymorphisms, then a particular member containing both such alleles would have a very high probability of exhibiting the cardiovascular condition.

The detection of multiple polymorphic sites permits one to define the frequency with which such sites independently segregate in a population. If, for example, two polymorphic sites segregate randomly, then they are either on separate chromosomes, or are distant to one another on the same chromosome. Conversely, two polymorphic sites that are co-inherited at significant frequency are linked to one another on the same chromosome. An analysis of the frequency of segregation thus permits the establishment of a genetic map of markers. Thus, the present invention provides a means for mapping the genomes of plants and animals.

The resolution of a genetic map is proportional to the number of markers that it contains. Since the methods of the present invention can be used to isolate a large number of polymorphic sites, they can be used to create a map having any desired degree of resolution.

The sequencing of the polymorphic sites greatly increases their utility in gene mapping. Such sequences can be used to design oligonucleotide primers and probes that can be employed to “walk” down the chromosome and thereby identify new marker sites (Bender, W. et al., J. Supra. Molec. Struc. 10(Supp.):32 (1979); Chinault, A. C. et al., Gene 5:111-126 (1979); Clarke, 10 L. et al., Nature 287:504-509 (1980)).

The resolution of the map can be further increased by combining polymorphic analyses with data on the phenotype of other attributes of the plant or animal whose genome is being mapped. Thus, if a particular polymorphism segregates with brown hair color, then that polymorphism maps to a locus near the gene or genes that are responsible for hair color. Similarly, biochemical data can be used to increase the resolution of the genetic map. In this embodiment, a biochemical determination (such as a serotype, isoform, etc.) is studied in order to determine whether it co-segregates with any polymorphic site. Such maps can be used to identify new gene sequences, to identify the causal mutations of disease, for example.

Indeed, the identification of the SNPs of the present invention permits one to use complimentary oligonucleotides as primers in PCR or other reactions to isolate and sequence novel gene sequences located on either side of the SNP. The present invention includes such novel gene sequences. The genomic sequences that can be clonally isolated through the use of such primers can be transcribed into RNA, and expressed as protein. The present invention also includes such protein, as well as antibodies and other binding molecules capable of binding to such protein.

The invention is illustrated below with respect to two of its embodiments —horses and humans. However, because the fundamental tenets of genetics apply irrespective of species, such illustration is equally applicable to any other species. Those of ordinary skill would therefore need only to directly employ the methods of the above invention to isolate SNPs in any other species, and to thereby conduct the genetic analysis of the present invention.

As indicated above, LOD scoring methodology has been developed to permit the use of RFLPs to both track the inheritance of genetic traits, and to construct a genetic map of a species (Lander, S. et al., Proc. Natl. Acad. Sci. (U.S.A.) 83:7353-7357 (1986); Lander, S. et al., Proc. Natl. Acad. Sci. (U.S.A.) 5 84:2363-2367 (1987); Donis-Keller, H. et al., Cell 51:319-337 (1987); Lander, S. et al., Genetics 121:185-199 (1989)). Such methods can be readily adapted to permit their use with the polymorphisms of the present invention. Indeed, such polymorphisms are superior to RFLPs and STRs in this regard. Due to the frequency of SNPs, it is possible to readily generate a dense genetic map. Moreover, as indicated above, the polymorphisms of the present invention are more stable than typical VNTR-type polymorphisms.

The polymorphisms of the present invention comprise direct genomic sequence information and can therefore be typed by a number of methods. In an RFLP or STR-dependent map, the analysis must be gel-based, and entail obtaining an electrophoretic profile of the DNA of the target animal. In addition to gel-based methods, an analysis of the polymorphisms (SNPs) may be performed using spectrophotometric methods, and can readily be automated to facilitate the analysis of large numbers of target animals.

Having now generally described the invention, the same will be more readily understood through reference to the following examples of the isolation and analysis of equine polymorphisms which are provided by way of illustration, and are not intended to be limiting of the present invention.

### EXAMPLE 1

### ANALYSIS OF MULTIPLE SNPs BY POLYACRYLAMIDE GEL ELECTROPHORESIS

In this example of the detection of multiple single nucleotide polymorphisms, a single stranded DNA template is probed with three interrogation primers. Primer #1 has a 5 base T-tail, primer #2 has a 10 base T-tail, and primer #3 has a 15 base T-tail.

To obtain single-stranded template, either of two methods may be used. First, the amplification may be mediated using primers that contain 4 phosphorothioate-nucleotide derivatives, as taught by Nikiforov, T. (U.S. patent application Ser. No. 08/005,061 (application was abandoned Jun. 24, 1996)). Alternatively, a second round of PCR may be performed using “asymmetric” primer concentrations. The products of the first reaction are diluted 1/1000 in a second reaction. One of the second round primers is used at the standard concentration of 2 M while the other is used at 0.08 M. Under these conditions, single stranded molecules are synthesized during the reaction.

The primer mixture is hybridized to the single stranded target template and a single base extension reaction using DNA polymerase and the four modified non-extendible nucleotides is allowed to occur. For each reaction tube, only one modified non-extendible nucleotide is labeled, preferably with ³²P or a florescent molecule. The resultant extended primers are then separated for analysis on a standard 12% sequencing gel. Thus, it is possible to determine the identity of the SNP corresponding to each primer based upon its electrophoretic mobility and the identity of the labeled non-extendible nucleotide.

### EXAMPLE 2

### ANALYSIS OF MULTIPLE SNPS BY SIZE EXCLUSION CHROMATOGRAPHY

Primers 1, 2, 3 and 4 are covalently coupled to BSA, ovalbumin, lysozyme and CIP, respectively. A single stranded DNA template is probed with the four interrogation primers and the single base extension reaction using DNA polymerase and the four modified non-extendible nucleotides is allowed to occur. Preferably each non-extendible nucleotide is uniquely and distinctly labeled.

The resultant extended primers are subsequently separated over a suitable size exclusion column (e.g., sephadex, sepharose, etc.) and the eluate is analyzed (e.g., with a scintillation counter) to determine the identity of the incorporated nucleotide.

### EXAMPLE 3

### ANALYSIS OF MULTIPLE SNPS USING AFFINITY TECHNIQUES

For this experiment, a peptide or protein affinity ligand is covalently coupled to the interrogation oligonucleotide using, for example, the methods disclosed by Chu et al., (Nucleic Acids Res. 16: 3671-3691 (1988)), herein incorporated by reference. The affinity ligand-interrogation primer complex is then hybridized to the target nucleic acid molecule and the single base extension reaction, described above, is allowed to occur in the presence of four differentially labeled dideoxynucleotide species (ddA, ddT, ddC, and ddG). Where desired, fewer species of dideoxynucleotides may be employed.

The corresponding monoclonal antibody to the peptide or protein is immobilized to a microtiter plate (Nunc). Each monoclonal antibody is immobilized to the microtiter plate at room temperature in a buffered solution. The plate is then washed with a TNTw solution three times to remove any excess unbound proteins.

Then the extended primer solution is added to each well for approximately 30 minutes. Unbound primer is removed by extensive washing with a TNTw solution. Table 3 is illustrative of the results that would be expected from such an experiment.

TABLE 3

| | Primer 1 | Primer 2 | Primer 3 |
|---|---|---|---|
| G | ND | ND | + |
| A | ND | + | ND |
| T | + | ND | ND |
| C | ND | ND | ND |

### EXAMPLE 4

### ANALYSIS OF MULTIPLE SNPS WITHOUT SEPARATING THE OLIGONUCLEOTIDES

It is also possible to identify multiple single nucleotide polymorphisms without separating the oligonucleotide probes. In such an experiment, each dideoxynucleotide triphosphate is preferably uniquely labeled.

Thus, for example, ddATP could be labeled with ³²P, ddGTP labeled with ³H, ddCTP labeled with ³⁵S, and ddTTP labeled with ¹²⁵I. Table 3 illustrates the hypothetical results obtained from hybridization with 6 oligonucleotides hybridized to a preparation containing nucleic acids of interest, and the result of a single base extension reaction. In such an experiment, the unincorporated ddNTP's may be separated from the extended probes using any of a variety of means (e.g., suitable spin column (i.e., CentriSep spin columns), etc.).

The labeled incorporated dideoxynucleotide triphosphates are subsequently detected using a scintillation counter. As each isotope has a distinct emission spectra, the scintillation counter can determine the identity of multiple single nucleotide polymorphisms without the need for purification procedures.

TABLE 4

| | Primers 1, | Primers 2, | Primers 3, | Primers 4, |
|---|---|---|---|---|
| | 2 and 3 | 3 and 4 | 4 and 5 | 5 and 6 |
| ddGTP | + | + | ND | + |
| ddATP | + | + | + | ND |
| ddCTP | ND | ND | + | + |
| ddTTP | + | + | + | + |

As depicted in Table 4, it is evident that identity of the incorporated dideoxynucleotide complementary to the single nucleotide polymorphism with respect to primers 1-6 are T, G, A, T, C, G, respectively. Any ambiguity can be determined by changing the combination of the primers.

### EXAMPLE 5

### ANALYSIS OF MULTIPLE SNPS BY OLIGONUCLEOTIDE ARRAY SEPARATION

Primer 1, 2, 3 and 4 contain in addition to sequences complimentary to the template DNA, unique sequences for subsequent hybridization to capture oligonucleotides on a solid surface. A single stranded DNA template is probed with the four interrogation primers and the single base extension reaction using DNA polymerase and the four non-extendible nucleotides is allowed to occur. Preferably each non-extendible nucleotide is uniquely and distinctly labeled.

The resultant extended primers are subsequently applied to the surface of an oligonucleotide array. The array consists of four separate and spatially distinct capture oligonucleotides, each of which is complimentary to a unique sequence on one of the interrogation primers. Each interrogation primer is effectively separated by hybridization to its corresponding surface bound capture primer. The identity of the labeled nucleotides is then determined by suitable methods.

While the invention has been described in connection with specific embodiments thereof, it will be understood that it is capable of further modifications and this application is intended to cover any variations, uses, or adaptions of the invention following, in general, the principles of the invention and including such departures from the present disclosure as come within known or customary practice within the art to which the invention pertains and as may be applied to the seesntial features hereinbefore set forth and as follows in the scope of the appended claims.

What is claimed is:

1. A method for detecting one or more single polymorphisms in a single reaction comprising the steps:
A) hybridizing one or more distinguishable interrogation oligonucleotide primers to one or more target nucleic acid molecules wherein each oligonucleotide primer is complementary to a specific and unique region of each target nucleic acid molecule such that the 3′ end of each primer is immediately proximal to a specific and unique target nucleotide of interest;
B) extending each interrogation oligonucleotide with a template-dependent polymerase wherein said extension occurs in the presence of one or more non-extendible nucleotide or nucleotide analog species; and
C) determining the identity of each nucleotide of interest by determining, for each interrogation primer employed, the identity of the non-extendible nucleotide (or nucleotide analog) incorporated into such primer, said identified non-extendible nucleotide or nucleotide analog being complementary to said primer's target nucleotide.

2. The method according to claim 1 wherein each interrogation oligonucleotide primer comprises a 5′ tail, said 5′ tail is composed of a neutral component having a specific and unique length or other characteristics used to identify or separate each interrogation primer.

3. The method according to claim 1 wherein said hybridization step occurs in solution.

4. The method according to claim 1 wherein the non-extendible nucleotide is identified by physical or chemical methods.

5. The method according to claim 4 werein the physical or chemical methods are selected from the group consisting of polarization spectroscopy, mass spectroscopy, infra-red spectroscopy, ultra-violet spectroscopy, visible spectroscopy or NMR spectroscopy.

6. The method according to claim 1 further comprising the step:
D) separating said extended primers on a suitable matrix.

7. The method according to claim 6 wherein said matrix is a size separating matrix.

8. The method according to claim 7 wherein said size separating matrix is a sequencing gel.

9. The method according to claim 8 wherein said sequencing gel contains from about 4% to about 20% polyacrilamide.

10. The method according to claim 7 wherein said size separating matrix is a size exclusion column.

11. The method according to claim 6 wherein said suitable receptor molecule is coupled to said matrix and wherein said suitable ligand molecule, corresponding to said receptor molecule, is coupled to said oligonucleotide primer.

12. The method according to claim 11 wherein said matrix is selected from the group consisting of a bead, a column, a dipstick, a microtiter plate, and a glass slide.

13. The method according to claim 1 wherein said non-extendible nucleotide is a ddNTP.

14. The method according to claim 13 wherein said ddNTP is fluorescently or chemically labeled.

15. The method according to claim 13 wherein said ddNTP is biotinylated.

16. The method according to claim 1 wherein said target molecule is a nucleic acid molecule.

17. The method according to claim 16 wherein said nucleic acid molecule is a DNA molecule.

18. The method according to claim 17 wherein said DNA molecule is genomic DNA.

19. The method according to claim 17 wherein said DNA molecule is double-stranded DNA.

20. The method according to claim 17 wherein said DNA molecule is single-stranded DNA.

21. The method according to claim 16 wherein said nucleic acid molecule is an RNA molecule.

22. A method for characterizing a target DNA comprising the steps:
A) hybridizing one or more of distinguishable interrogation oligonucleotide primers to one or more target nucleic acdd molecules wherein each oligonucleotide primer is complementary to a specific and unique region of each target nucleic acid molecule such that the 3′ end of each primer is immediately proximal to a specific and unique target nucleotide of interest;
B) extending each interrogation oligonucleotide with a template-dependent polymerase wherein said extension occurs in the presence of more than one non-extendible nucleotide species;
C) separating said extended primers on a suitable matrix;
D) interrogating each nucleotide of interest by determining, for each interrogation primer employed, the identity of the non-extendible nucleotide incorporated into such primer, said identified non-extendible nucleotide being complementary to said primer's target nucleotide; and
(E) comparing said interrogated nucleotide of interest of said target, with a corresponding nucleotide of interest of a reference nucleic acid molecule, and determining whether said nucleotides of interest contain the same single nucleotide at their respective sites.

23. The method according to claim 22 wherein said characterization identifies a trait of said target DNA molecule.

24. The method according to claim 23 wherein said trait is a genetic disease.

25. The method according to claim 23 wherein said trait is a genetic condition.

26. The method according to claim 7 wherein the size separating matrix is selected from the group consisting of sepharose and sephadex.

27. The method according to claim 11 wherein the ligand is selected from the group consisting of a hapten, an antigen, a cofactor, biotin, and iminobiotin.

28. The method according to claim 11 wherein the ligand is selected from the group consisting of dinitrophenol, lipoic acid, and an olefinic compound.

29. The method according to claim 11 wherein the ligand is selected from the group consisting of unique and specific oligonucleotides designed to hybridize specifically to complementary oligonucleotides, PNA sequences designed to hybridize specifically to complementary oligonucleotides and PNA sequences that function as receptors.

30. The method according to claim 11 wherein the ligand is selected from the group consisting of an antibody, an enzyme, a polypeptide, strepavidin, and avidin.

31. The method according to claim 11 wherein the ligand is capable of forming a complex by binding with a detectable polypeptide.

32. The method according to claim 30 wherein the detectable polypeptide is selected from the group consisting of an antibody, an enzyme capable of depositing insoluble reaction products, streptavidin, and avidin.

33. The method according to claim 30 wherein the detectable polypeptide is selected from randomly generated polypeptide libraries.

34. The method according to claim 11 wherein the receptor is selected from the group consisting of a hapten, an antigen, a cofactor, biotin, and iminobiotin.

35. The method according to claim 11 wherein the receptor is selected from the group consisting of dinitrophenol, lipoic acid, and an olefinic compound.

36. The method according to claim 11 wherein the receptor is selected from the group consisting of unique and specific oligonucleotides designed to hybridize specifically to complementary oligonucleotides, PNA sequences designed to hybridize specifically to complementary oligonucleotides and PNA sequences that function as ligands.

37. The method according to claim 11 wherein the receptor is capable of forming a complex by binding with a detectable polypeptide.

38. The method according to claim 37 wherein the detectable polypeptide is selected from the group consisting of an antibody, an enzyme capable of depositing insoluble reaction products, streptavidin, and avidin.

39. The method according to claim 37 wherein the detectable polypeptide is selected from randomly generated polypeptide libraries.

40. The method according to claim 11 wherein the receptor molecule is coupled to the matrix by methods selected from the group consisting of covalent coupling, ionic interactions, non-specific adsorption, and specific, but non-covalent ligand-receptor interactions.

41. The method according to claim 40 wherein the ligand-receptor is selected from complimentary hybridizing nucleic acids.

42. The method according to claim 40 wherein the ligand-receptor is selected from the group consisting of complimentary hybridizing PNAs and other synthetic nucleic acid analogs.

43. The method according to claim 11 wherein the ligand molecule is coupled to the oligonucleotide primer by methods selected from the group consisting of covalent coupling, ionic interactions, non specific adsorption, and specific but non-covalent ligand-receptor interactions.

44. The method according to claim 43 wherein the ligand-receptor is selected from the group consisting of complimentary hybridizing nucleic acids.

45. The method according to claim 43 wherein the ligand-receptor is selected from the group consisting of complimentary hybridizing PNAs or other synthetic nucleic acid analogs.

46. The method according to claim 1 wherein said non-extendible nucleotide is a synthetic or naturally occurring nucleotide analog that is able to be incorporated by a template dependent polymerase.

47. The method according to claim 46 wherein said synthetic or naturally occurring nucleotide analog is selected from the group consisting of acyclic ribose, substituted nucleotide analogs, and modified ribose nucleotide analogs.

48. The method according to claim 46 wherein said synthetic nucleotide analog is selected from the group consisting of fructose based nucleotide analog.

49. The method according to claim 46 wherein said synthetic nucleotide analog is selected from the group consisting of chemically modified purine or pyrimidine that retains the ability to specifically base pair with naturally occurring nucleotides.

50. The method according to claim 46 wherein said synthetic nucleotide analog is selected from the group consisting of compound that retains the ability to specifically base pair with naturally occurring nucleotides.

51. The method according to claim 1 wherein said non-extendible nucleotide is fluorescently or chemically labeled.

52. The method according to claim 1 wherein said non-extendible nucleotide is labeled with biotin or iminobiotin.

53. The method according to claim 1 wherein said non-extendible nucleotide is labeled with a hapten, an antigen or a cofactor.

54. The method according to claim 1 wherein said non-extendible nucleotide is labeled with dinitrophenol, lipoic acid, or an olefinic compound.

55. The method according to claim 1 wherein said non-extendible nucleotide is labeled with a detectable polypeptide.

56. The method according to claim 1 wherein said non-extendible nucleotide is labeled with a molecule that is electron dense or an enzyme capable of depositing an insoluble reaction product.

57. The method according to claim 1 wherein said non-extendible nucleotide is labeled with a molecule that is electron dense or an enzyme capable of depositing an insoluble reaction product.

58. The method of claim 48 wherein the fluorescent indicator molecule is selected from the group consisting of fluorescein, rhodamine, texas red, FAM, JOE, TAMRA, ROX, HEX, TET, Cy3, Cy3.5, Cy5, Cy5.5, IRD40, IRD41 and BODIPY.

59. The method of claim 57 wherein the electron dense indicator molecule is selected from the group consisting of ferritin, hemocyanin, and colloidal gold.

60. The method of claim 55 wherein the detectable polypeptide is indirectly detectable by specifically complexing the detectable polypeptide with a second polypeptide covalently linked to an indicator molecule.

61. The method of claim 60 wherein said detectable polypeptide is selected from the group consisting of avidin and streptavidin and the second polypeptide is selected from the group consisting of biotin and iminobiotin.

62. The method according to claim 16 wherein said nucleic acid molecule is from a plant.

63. The method according to claim 16 wherein said nucleic acid molecule is from a microorganism.

64. The method according to claim 63 wherein said microorganism is selected from the group consisting of bacteria, fungi, yeast, viruses, viroids and other heritable genetic entity.


SEQUENCE LISTING (72 sequences)

SEQ ID NO: 1 - DNA, 20 bp, Equus caballus
gcagctctaagtgctgtggg

SEQ ID NO: 2 - DNA, 20 bp, Equus caballus
tgcagaaattctaaggtgtt

SEQ ID NO: 3 - DNA, 20 bp, Equus caballus
aacaccttagaatttctgca

SEQ ID NO: 4 - DNA, 20 bp, Equus caballus
cccacagcacttagagctgc

SEQ ID NO: 5 - DNA, 20 bp, Equus caballus
agctctgggatgatccacta

SEQ ID NO: 6 - DNA, 20 bp, Equus caballus
tgagggaaaaatgatgatgc

SEQ ID NO: 7 - DNA, 20 bp, Equus caballus
gcatcatcatttttccctca

SEQ ID NO: 8 - DNA, 20 bp, Equus caballus
tagtggatcatcccagagct

SEQ ID NO: 9 - DNA, 20 bp, Equus caballus
aaaactaatttgatggccat

SEQ ID NO: 10 - DNA, 20 bp, Equus caballus
aaagtcagaacaatgattgc

SEQ ID NO: 11 - DNA, 20 bp, Equus caballus
gcaatcattgttctgacttt

SEQ ID NO: 12 - DNA, 20 bp, Equus caballus
atggccatcaaattagtttt

SEQ ID NO: 13 - DNA, 20 bp, Equus caballus
cacaaggcccaagaacagga

SEQ ID NO: 14 - DNA, 20 bp, Equus caballus
tgagttcagcgagtgtcaga

SEQ ID NO: 15 - DNA, 20 bp, Equus caballus
tctgacactcgctgaactca

SEQ ID NO: 16 - DNA, 20 bp, Equus caballus
tcctgttcttgggccttgtg

SEQ ID NO: 17 - DNA, 20 bp, Equus caballus
tgggaaagaccacattattt

SEQ ID NO: 18 - DNA, 20 bp, Equus caballus
gttcccttttgtttcagacc

SEQ ID NO: 19 - DNA, 20 bp, Equus caballus
ggtctgaaacaaaagggaac

SEQ ID NO: 20 - DNA, 20 bp, Equus caballus
aaataatgtggtctttccca

SEQ ID NO: 21 - DNA, 20 bp, Equus caballus
catgagtaagaagcatccgg

SEQ ID NO: 22 - DNA, 20 bp, Equus caballus
ccatggagtcatagataagt

SEQ ID NO: 23 - DNA, 20 bp, Equus caballus
acttatctatgactccatgg

SEQ ID NO: 24 - DNA, 20 bp, Equus caballus
ccggatgcttcttactcatg

SEQ ID NO: 25 - DNA, 20 bp, Equus caballus
cccaagaacaggattgagtt

SEQ ID NO: 26 - DNA, 20 bp, Equus caballus
agcgagtgtcagagttgtgt

SEQ ID NO: 27 - DNA, 20 bp, Equus caballus
acacaactctgacactcgct

SEQ ID NO: 28 - DNA, 20 bp, Equus caballus
aactcaatcctgttcttggg

SEQ ID NO: 29 - DNA, 20 bp, Equus caballus
agcaagaaatggggggcctt

SEQ ID NO: 30 - DNA, 20 bp, Equus caballus
gtcctacaattgccaggaag

SEQ ID NO: 31 - DNA, 20 bp, Equus caballus
cttcctggcaattgtaggac

SEQ ID NO: 32 - DNA, 20 bp, Equus caballus
aaggccccccatttcttgct

SEQ ID NO: 33 - DNA, 20 bp, Equus caballus
gaatatcaatatatatatat

SEQ ID NO: 34 - DNA, 20 bp, Equus caballus
tgtgtgtgtgtgtatttgct

SEQ ID NO: 35 - DNA, 20 bp, Equus caballus
agcaaatacacacacacaca

SEQ ID NO: 36 - DNA, 20 bp, Equus caballus
atatatatatattgatattc

SEQ ID NO: 37 - DNA, 20 bp, Equus caballus
gccataattaagcctgtatt

SEQ ID NO: 38 - DNA, 20 bp, Equus caballus
gtttgttttaaattttgtga

SEQ ID NO: 39 - DNA, 20 bp, Equus caballus
tcacaaaatttaaaacaaac

SEQ ID NO: 40 - DNA, 20 bp, Equus caballus
aatacaggcttaattatggc

SEQ ID NO: 41 - DNA, 20 bp, Equus caballus
gtgtagagtagttcaaggac

SEQ ID NO: 42 - DNA, 20 bp, Equus caballus
atgtcttatacctccctttt

SEQ ID NO: 43 - DNA, 20 bp, Equus caballus
aaaagggaggtataagacat

SEQ ID NO: 44 - DNA, 20 bp, Equus caballus
gtccttgaactactctacac

SEQ ID NO: 45 - DNA, 20 bp, Equus caballus
gtgaacggagagcaggcctt

SEQ ID NO: 46 - DNA, 20 bp, Equus caballus
cctgctgaagcctcagaccg

SEQ ID NO: 47 - DNA, 20 bp, Equus caballus
cggtctgaggcttcagcagg

SEQ ID NO: 48 - DNA, 20 bp, Equus caballus
aaggcctgctctccgttcac

SEQ ID NO: 49 - DNA, 20 bp, Equus caballus
ctgctctttagactatgacc

SEQ ID NO: 50 - DNA, 20 bp, Equus caballus
tcaaccttgcatcatgagct

SEQ ID NO: 51 - DNA, 20 bp, Equus caballus
agctcatgatgcaaggttga

SEQ ID NO: 52 - DNA, 20 bp, Equus caballus
ggtcatagtctaaagagcag

SEQ ID NO: 53 - DNA, 20 bp, Equus caballus
tttgagctgggacctcagtc

SEQ ID NO: 54 - DNA, 20 bp, Equus caballus
tctcctgcctttagactcga

SEQ ID NO: 55 - DNA, 20 bp, Equus caballus
tcgagtctaaaggcaggaga

SEQ ID NO: 56 - DNA, 20 bp, Equus caballus
gactgaggtcccagctcaaa

SEQ ID NO: 57 - DNA, 20 bp, Equus caballus
gaacctctgggccgtggata

SEQ ID NO: 58 - DNA, 20 bp, Equus caballus
ttgttcagaagcacaggtga

SEQ ID NO: 59 - DNA, 20 bp, Equus caballus
tcacctgtgcttctgaacaa

SEQ ID NO: 60 - DNA, 20 bp, Equus caballus
tatccacggcccagaggttc

SEQ ID NO: 61 - DNA, 20 bp, Equus caballus
gtatttgctagctctgggat

SEQ ID NO: 62 - DNA, 20 bp, Equus caballus
atccactaatgagggaaaaa

SEQ ID NO: 63 - DNA, 20 bp, Equus caballus
tttttccctcattagtggat

SEQ ID NO: 64 - DNA, 20 bp, Equus caballus
atcccagagctagcaaatac

SEQ ID NO: 65 - DNA, 20 bp, Equus caballus
gaagttgtgggacagatgtg

SEQ ID NO: 66 - DNA, 20 bp, Equus caballus
agagatgcagctctaagtgc

SEQ ID NO: 67 - DNA, 20 bp, Equus caballus
gcacttagagctgcatctct

SEQ ID NO: 68 - DNA, 20 bp, Equus caballus
cacatctgtcccacaacttc

SEQ ID NO: 69 - DNA, 20 bp, Equus caballus
ccatgaggaagcctccacaa

SEQ ID NO: 70 - DNA, 20 bp, Equus caballus
gtcccaatagtctgggattc

SEQ ID NO: 71 - DNA, 20 bp, Equus caballus
gaatcccagactattgggac

SEQ ID NO: 72 - DNA, 20 bp, Equus caballus
ttgtggaggcttcctcatgg